SEQUENCE LISTING (3 sequences)

SEQ ID NO: 1 - DNA, 2196 bp, Homo sapiens
ctgggcgggtcatgcgccctggccttcgcgcatctcccaggttagctgcgtgtccgggtgctaggctgcagacccgccgccatgacgctgcgcgcggccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctccggccctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtgtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgactatgctgctggctgtctttgttcttggagaggtggagtgactgttcacggagaa

SEQ ID NO: 2 - protein, 555 aa, Homo sapiens
MTLRAAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGSGPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 3 - DNA, 23 bp, Artificial Sequence
  Description of Artificial Sequence Synthetic oligonucleotide
aannnnnnnnnnnnnnnnnnntt

(19) United States
(12) Patent Application Publication
(10) Pub. No.: US 2012/0315283 A1
(43) Pub. Date: Dec. 13, 2012

(54) METHODS OF PROMOTING TISSUE GROWTH AND TISSUE REGENERATION

(75) Inventors: Dipak Panigraphy, Boston, MA (US); Mark Kieran, Newton, MA (US)

(73) Assignees: DANA-FARBER CANCER INSTITUTE, INC., Boston, MA (US); CHILDREN'S MEDICAL CENTER CORPORATION, Boston, MA (US)

(21) Appl. No.: 13/521,163

(22) PCT Filed: Feb. 1, 2011
(86) PCT No.: PCT/US11/23329

§ 371 Date: Aug. 14, 2012

Related U.S. Application Data
(60) Provisional application No. 61/300,477, filed on Feb. 2, 2010.

(51) Int. Cl. C12N 5/071; A61K 31/7088; A61K 38/17; A61P 9/00; A61K 31/192; A61K 31/4468; A61P 25/00; A61K 31/713; A61K 39/395

(52) U.S. Cl. 424/158.1; 435/375; 514/44 A; 514/44 R; 514/1.1; 514/564; 514/329; 514/13.3

(57) ABSTRACT

Described herein are methods of using soluble epoxide hydrolase inhibitors to modulate the levels of epoxyeicosatrienoic acids (EETs) in order to increase angiogenesis and promote wound healing and tissue regeneration.

### CROSS REFERENCE TO RELATED APPLICATIONS

This application claims benefit under 35 U.S.C. §119(e) of U.S. provisional application 61/300,477 filed Feb. 2, 2010, which is incorporated herein by reference in its entirety.

### GOVERNMENT SUPPORT

The present invention was made with government support under Grant No. Z01 025034 awarded by the National Institute of Health. The government has certain rights in this invention.

### FIELD OF THE INVENTION

The field of the invention relates to wound healing and tissue and/or organ regeneration. As more particularly provided herein, the field of the invention relates to promoting wound healing and tissue and/or organ regeneration with soluble epoxide hydrolase inhibitors.

### BACKGROUND OF INVENTION

The majority of the millions of plastic and reconstructive surgical procedures performed each year are to repair soft tissue injuries that result from traumatic injury (i.e., significant burns), tumor resection (i.e., mastectomy and carcinoma removal), and congenital defects. Numerous in vivo factors can influence a body's innate ability to repair damaged tissue and/or organs. One major factor is adequate blood supply to the area needing repair because adequate blood supply ensures that the necessary repair materials and growth factors are made available at the repair site. Adequate blood supply allows the body to heal and repair itself by increasing cell proliferation and promoting tissue growth.

Angiogenesis is the formation, development and growth of new blood vessels. The normal regulation of angiogenesis is governed by a fine balance between factors that induce the formation of blood vessels and those that halt or inhibit the process. Promoting angiogenesis would provide an adequate blood supply for tissue and/or organ regeneration and wound healing.

Many factors modulate angiogensis. One class of these molecules are EETs, four of which (5,6-EET, 8,9-EET, 11,12-EET and 14,15-EET) have been investigated as autocrine and paracrine mediators of arachidonic acid-induced vasorelaxation in the cardiovascular and renal system. EETs play a role in tissue homeostasis which results from their effects on cellular proliferation, migration and inflammation. Blood vessels also represent a major target of EETs, which have been shown to stimulate angiogenesis. EETs are produced from arachidonic acid by cytochrome P450 (CYP) epoxygenases CYP2C8 and CYP2J2 and mainly metabolized by soluble epoxide hydrolase (sEH), also known as EPHX2, to less active dihydroxyeicosatrienoic acids (DHETs). Inhibitors of sEH, which raise endogenous EET levels, are in clinical trials as anti-hypertensive agents.

### SUMMARY OF THE INVENTION

Embodiments of the present invention are based on the discoveries that sEH inhibitors increase the plasma levels of epoxyeicosatrienoic acids. The inventors found that their modulation of lipid mediator concentrations encourages angiogenesis, cell proliferation, wound healing, organ regeneration, and microvessel density. Methods comprising administering sEH inhibitors to a tissue or a patient are therefore useful in promoting angiogenesis, such as in wound healing, tissue repair, fertility treatments, hypertrophied hearts, revascularization of tissue after disease and trauma (e.g. stroke, ischemic limbs, vascular diseases, bone repair), tissue grafts, tissue engineered constructs, and treating erectile dysfunction. In one embodiment, the invention comprising the method of administering a sEH inhibitor where the sEH inhibitor is t-AUCB or TUPS.

In one embodiment, described herein is a composition comprising a pharmaceutically acceptable carrier and a sEH inhibitor.

In one embodiment, a method or use for promoting cell proliferation, angiogenesis, tissue growth, or tissue regeneration in a tissue in need thereof is provided, the method comprising contacting the tissue with a composition comprising a sEH inhibitor, e.g. in wound healing, tissue repair, and/or bone grafts.

In one embodiment, one first identifies a tissue in need of e.g., cell proliferation and then contacts the tissue with a sEH inhibitor, as that term is used herein.

In one embodiment, a method of promoting angiogenesis in a tissue in need thereof is provided, the method comprising contacting the tissue with a composition comprising a sEH inhibitor. The method is applied in the context of, but is not limited to, wound healing, tissue repair, impaired fertility, cardiac hypertrophy, erectile dysfunction, bone healing, promoting revascularization after disease or trauma, tissue grafts, or tissue engineered constructs.

Accordingly, in one embodiment, one first diagnosis the individual has having a wound in need of healing, or tissue in need of repair, impaired fertility, cardiac hypertrophy, erectile dysfunction, tissue in need of revascularization, tissue in need of grafting or engineered constructs and then contacts the tissue with a sEH inhibitor as described herein.

In one embodiment of this aspect and all other aspects described herein, the sEH inhibitor is t-AUCB (trans-4-[4-(3-adamantan-1-yl-ureido)-cyclohexyloxy]-benzonic acid).

In one embodiment of this aspect and all other aspects described herein, the sEH inhibitor is TUPS 1-(1-methanesulfonyl-piperidin-4-yl)-3-(4-trifluoromethoxy-phenyl)-urea.

In one embodiment, a sEH inhibitor is a ligand that specifically binds to sEH or a nucleic acid probe which reduces transcription of sEH (e.g., RNAi). In one embodiment of this aspect and all other aspects described herein, the sEH inhibitor is an antibody or nucleic acid probe specifically directed against sEH.

### DEFINITIONS

As used herein, the term “inhibit” or “inhibition” means the reduction or prevention of sEH enzyme activity or the reduction or prevention of sEH gene expression. In one embodiment, the inhibition is in a cell. In a preferred embodiment, the inhibition is in an endothelial cell. The reduction in activity or gene expression can be by about 5%, about 10%, about 20%, about 30%, about 40%, about 50%, about 60%, about 70%, about 80%, about 90%, about 100%, about 125%, about 150% or more compared to a control, which is activity in the absence of an inhibitor.

As used herein, a “sEH inhibitor” (sEHi) is an agent (e.g., small molecule, ligand or an antibody) which inhibits the activity or the expression of soluble epoxide hydrolase (sEH) gene by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a sEH inhibitor relative to in the absence of such agent.

In one embodiment, the inhibition of the expression of sEH gene is by RNA interference; the “sEH inhibitor” can be a nucleic acid probe capable of binding to a portion of the sEH mRNA. The complementary nucleic acid probe, as used herein, can be complementary to any portion of a sEH mRNA including sense and anti-sense strands of the gene, and including coding and non-coding sequences. Additionally, the sEH inhibitor will be capable of reducing transcription of sEH by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a sEH inhibitor relative to transcription in the absence of such agent.

The nucleic acid probe may consist of Sequence 1 or any derivative or fragment thereof. Design of nucleotide sequences capable of reducing transcription or translation of sEH will be obvious to those skilled in the art and may include, but are not limited to, RNAi, shRNA, miRNA, antisense oligonucleotides, siRNA, morpholinos and aptamers and may be RNA molecules, DNA molecules, or modified forms or analogs thereof. In certain embodiments, such nucleic acid probes would be double-stranded siRNA such as the products available from Santa Cruz Biotechnology as catalog #sc-44090. Means of delivering such nucleotide sequences to the target cells, tissue, or patient will also be obvious to those skilled in the art and include but are not limited to, delivery of oligonucleotides themselves, delivery by a vector, or delivery of a mixture comprising the oligonucleotide or vector and at least one other compound. Design and delivery of oligonucleotides are typified but not limited by the methods taught in Verreault, M., et al. Current Gene Therapy 2006, 6, 505-533, Lu, P. Y., et al. Trends in Molecular Medicine 2005, 11, 104-113, Huang, C. et al. Expert Opinion on Therapeutic Targets 2008, 12, 637, Cheema, S. K. et al., Wound Repair and Regeneration 2007, 15, 286, Khurana, B. et al., 2010, 10, 139, Shim, M. S, and Kwon, Y. J. FEBS J, 2010, 277, 4814, Walton, S. P., et al., FEBS J 2010, 277, 4806, Sliva, K. and Schnierle, B. S., Virology Journal 2010, 7; 248, Lares, M. R., et al. Trends in Biotechnology, 28, 570, Rossbach, M. Current Molecular Medicine, 2010, 10, 361, Pfiefer, A. and Lehmann, H. Pharmacology and Therapeutics 2010, 126, 217, Matthais, J. et al. (2003) “Gene Silencing by RNAi in Mammalian Cells” In Ausubel, F. M. et al. (Ed.) Current Protocols in Molecular Biology John Wiley & Sons, Inc.: Hoboken, N.J. These publications are hereby incorporated in their entirety by way of reference.

In one embodiment, the inhibition of the expression of sEH gene is by binding of a antibody which specifically recognizes an epitope of sEH. Additionally, this sEH inhibitor will decrease the activity of sEH by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a sEH inhibitor relative to activity in the absence of such agent. The activity of sEH can be determined by a change in at least one measurable marker of sEH activity that is known in the art (e.g., the level of EET in an endothelial cell as described herein).

The level of sEH expression can be determined by any method that is known in the art, e.g., by western blot analysis of the sEH protein level.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

In one embodiment, the sEHi is one of the following compounds; t-AUCB, TUPS, entA-2b (Shen et al., Bioorg Med Chem Lett 2009 19:5314-20), AUDA (12-(3-adamantan-1-yl-ureido) dodecanoic acid) (Simpkins et al. Am J Pathol 2009 174:2086-95), compounds 27 and 28 as disclosed in Kasagami et al. Bioorg Med Chem Lett 2009, 19:1784-89, nbAUDA (the n-butyl ester of 12-(3-adamantan-1-yl-ureido)-dodecanoic acid) (Parrish et al Cell Biol Toxicol. 2009 25:217-25), compound 2 as disclosed in Morrisseau et al., Bioorg Med Chem Lett 2006 16:5439-5444, 4-(3-cyclohexylureido)-ethanoic acid (CU2), 4-(3-cyclohexylureido)-butyric acid (CU4), 4-(3-cyclohexylureido)-hexanoic acid (CU6), and 4-(3-cyclohexylureido)-heptanoic acid (CU7) (Gomez et al., Protein Sci 2006 15:58-64), 1-cyclohexyl-3-dodecylurea, 12-(3-cyclohexyl-ureido) dodecanoic acid and 950 [adamantan-1-yl-3-{5-[2-(2-ethoxyethoxy)ethoxy]pentyl}urea] (Olearczyk et al. J Pharmacol Exp Ther. 2006 318:1307-14), N-(1-Acetylpiperidin-4-yl)-N′-(adamant-1-yl)urea (5a) (Jones et al., Bioorg Med Chem Lett 2006 16:5212-6), 1,3-dicyclohexylurea (DCU), (Ghosh et al., Basic Clin Pharmacol Toxicol 2008 102:453-8, 1-adamantan-3-(5-(2-(2-ethylethoxy)ethoxy)pentyl)urea (AEPU) (Ulu et al., J Cardiovasc Pharmacol 2008 52:314-23), polyethylene glycol ester of AUDA, and 1-adamantan-1-yl-3-(5-(2-(2-ethoxyethoxy)ethoxy)-pentyl)urea as disclosed in Fife et al., J Pharmacol Exp Ther 2008 327:707-15, 12-(3-adamantan-1-yl-ureido)-dodecanoic acid butyl ester (AUDA-BE) (Motoki et al., Am J Physiol Heart Circ Physiol 2008 295:H2128-34, cis-4-[4-(3-adamantan-1-yl-ureido)-cyclohexyloxy]-benzoic acid (c-AUCB) (Jung et al., PLoS One 2010 5:e11979), 1-trifluoromethoxy-phenyl-3-(1-acetylpiperidin-4-yl)urea (TPAU) (Chiamnimonvat et al., J Cardiov Pharm 2007 50:225-237), and 1-(4-(4-(4-acetylpiperazin-1-yl)butoxy)phenyl)-3-adamantan-1-yl urea (Huang et al J Med Chem 2010 53: 8376-86).

In one embodiment, the sEHi is the compound AR9281 (1-(1-Acetyl-piperidin-4-yl)-3-adamantan-1-yl-urea), (Anandan et al., Bioorg Med Chem Lett 2011 21:983-8) used in the Phase II clinical trial “Evaluation of Soluble Epoxide Hydrolase (s-EH) Inhibitor in Patients with Mild to Moderate Hypertension and Impaired Glucose Tolerance” sponsored by Arete Therapeutics (US Government Clinical Trial ID: NCT00847899).

In one embodiment, the sEHi is the compound GSK2188931 (Kompa et al., European Heart Journal 2010 31:422 (Suppl.)) described in the clinical trial “Evaluation of the Effects of Urotensin-II and Soluble Epoxide Hydrolase Inhibitors on Skin Microvessel Tone in Patients With Heart Failure, and in Healthy Volunteers” sponsored by Monash University (US Government Clinical Trial ID: NCT00654966).

In one embodiment the sEHi is one of the compositions disclosed in one of the following publications, which are hereby incorporated by reference in their entirety: US 2010/0267807, US 2006/0293292, US 2010/0016310, US 2010/0074852, US 2006/0035869, US 2004/0092487, US 2007/0117782, US 2009/0215894, US 2008/0200444, US 2006/0276515, US 2009/0197916, US 2008/0221104, US 2009/0270382, US 2008/0200467, US 2009/0247521, US 2009/0270452, US 20009/0023731, US 2009/0099184, US 2008/0280904, Morrisseau et al., Bioorg Med Chem Lett 2006 16:5439-5444; Morisseau, C., et al. Biochemical Pharmacology 2002, 63, 1599; Jones, P. D., et al. Bioorganic & medicinal chemistry letters 2006, 16, 5212, Wolf, N. M. et al. Analytical Biochemistry 2006, 355, 71, Morisseau, C. et al. Proceedings of the National Academy of Sciences 1999, 96, 8849, Xie, Y., et al. Bioorganic & Medicinal Chemistry Letters 2009, 19, 2354, Anandan, S., et al. Bioorganic & Medicinal Chemistry Letters, 2009, 19, 1066, Eldrup, A. B. et al. Bioorganic & Medicinal Chemistry Letters 2010, 20, 571, Taylor, S. J. et al. Bioorganic & Medicinal Chemistry Letters 2009, 19, 5864, Kasagami, T. et al. Bioorganic & Medicinal Chemistry Letters 2009, 19, 1784, Anandan, S, and Gless, R. D. Bioorganic & Medicinal Chemistry Letters 2010, 20, 2740, Shen, H. C. et al. Bioorganic & Medicinal Chemistry Letters 2009, 19, 3398, Shen et al., Bioorg Med Chem Lett 2009 19:5716-21, Kim, et al. Bioorg Med Chem Lett 2007, 15:312-23, Kim et al., J Med Chem 2004 47:2110-22, Marino Curr Top Med Chem 2009 9:452-63, Qiu et al., Cardiovasc Ther 2010 E-pub, PMID: 20433684, Shen Expert Opin Ther Pat 2010 20:941-56, and Eldrup, A. B. et al. Journal of Medicinal Chemistry 2009, 52, 5880.

In one embodiment the sEHi is a 1) pyrazole phenyl derived amide, 2) N-substituted pridinone or pyrimidine derivative, 3) acyl hydrazone, 4) Aniline-derived amide, 5) Compound 61, 6) Benzimidazole-5-carboxamide, or 7) 3,3 disubstituted piperidine-derived urea.

As used herein, the term “t-AUCB” refers to a composition with the formulation of trans-4-[4-(3-adamantan-1-yl-ureido)-cyclohexyloxy]-benzonic acid (Hwang et al. J Med Chem. 2007; 50(16):3825-3840).

As used herein, the term “TUPS” refers to a composition with the formulation of 1-(1-methanesulfonyl-piperidin-4-yl)-3-(4-trifluoromethoxy-phenyl)-urea (Hwang et al. J Med Chem. 2007; 50(16):3825-3840).

As used herein, the term “therapeutically effective amount” refers to that amount of sEHi that can reduce the activity of a candidate protein by at least 5% or the expression of a sEH gene by at least 5%. The assays for determining activity or gene expression are described herein or other methods that are known to one skilled in the art can be used. In another embodiment, the term “therapeutically effective amount” refers to an increase of at least 5% in cell proliferation, angiogenesis, wound healing, tissue growth or regeneration compared to in the absence of the sEHi.

As used herein, the word “repair”, means the natural replacement of worn, torn or broken components with newly synthesized components. The word “healing”, as used herein, means the returning of torn and broken organs and tissues (wounds) to wholeness.

As used herein, the term “tissue regeneration” refers to the cell proliferation and cell growth in a tissue which aims to restore and repair tissue parts and function. In one embodiment, “tissue regeneration” encompasses the interplay of living cells, an extracellular matrix and cell communicators, e.g., growth factors, pro-angiogenic factors etc., to bring about cell proliferation and cell growth.

As used herein, the term “administering,” refer to the placement of the sEHi as disclosed herein into a subject by a method or route which results in at least partial localization of the agents at a desired site. The pharmaceutical compositions of comprising the sEHi disclosed herein can be administered by any appropriate route which results in an effective treatment in the subject.

As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” and “RNA interfering” with respect to an agent of the invention, are used interchangeably herein.

As used herein an “siRNA” refers to a nucleic acid that forms a double stranded RNA, which double stranded RNA has the ability to reduce or inhibit expression of a gene or target gene when the siRNA is present or expressed in the same cell as the target gene, sEH. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein, the term “complementary” or “complementary base pair” refers to A:T and G:C in DNA and A:U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

The term “vector”, as used herein, refers to a nucleic acid construct designed for delivery to a host cell or transfer between different host cells. As used herein, a vector can be viral or non-viral.

As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms, for example in human cells for expression and in a prokaryotic host for cloning and amplification.

As used herein, the term “heterologous nucleic acid fragments” refers to nucleic acid sequences that are not naturally occurring in that cell. For example, when a miR-150 gene is inserted into the genome of a bacteria or virus, that miR-150 gene is heterologous to that recipient bacteria or virus because the bacteria and viral genome do not naturally have the miR-150 gene.

As used herein, the term “viral vector” refers to a nucleic acid vector construct that includes at least one element of viral origin and has the capacity to be packaged into a viral vector particle. The viral vector can contain the sEH gene in place of non-essential viral genes. The vector and/or particle may be utilized for the purpose of transferring any nucleic acids into cells either in vitro or in vivo. Numerous forms of viral vectors are known in the art.

The term “replication incompetent” as used herein means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins from packaging the virus) and viral particles cannot be formed in the patient's cells.

The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “antibody” refers to immunoglobulin molecules and immunologically active portions of immunoglobulin molecules, i.e., molecules that contain an antigen binding site that immunospecifically bind an antigen. The terms also refers to antibodies comprised of two immunoglobulin heavy chains and two immunoglobulin light chains as well as a variety of forms besides antibodies; including, for example, Fv, Fab, and F(ab)′2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference).

As used herein, the term “pro-angiogenic activity” refers to the stimulation or enhancement of angiogenesis and/or endothelial cell proliferation.

As used herein, the terms “increasing angiogenesis”, “promoting angiogenesis” or “enhancing angiogenesis” refers to an increase in at least one measurable marker of angiogenesis by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a sEH inhibitor relative to that marker in the absence of such agent. For example, increase vascularization as described in the Example section.

Endothelial cell migration can be assessed, for example, by measuring the migration of cells through a porous membrane using a commercially available kit such as BD BioCoat Angiogenesis System or through a Boyden chamber apparatus. Thus, as used herein, the term “enhances cell migration” refers, at a minimum, to an increase in the migration of endothelial cells through a porous membrane of at least 10% in the presence of a sEH inhibitor; preferably the increase is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more in the presence of a sEHi, as that term is used herein.

Endothelial cell growth can be determined, for example, by measuring cell proliferation using an MTS assay commercially available from a variety of companies including RnD Systems, and Promega, among others. Thus, as used herein, the term “enhances cell proliferation” refers to an increase in the number of endothelial cells of at least 10% in the presence of a sEH inhibitor (as assessed using e.g., an MTS assay); preferably the increase is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more in the presence of a sEH inhibitor, as that term is used herein.

The term “wound” as used herein refers broadly to injuries to an organ or tissue of an organism that typically involves division of tissue or rupture of a membrane (e.g., skin), due to external violence, a mechanical agency, or infectious disease. The term “wound” encompasses injuries including, but not limited to, lacerations, abrasions, avulsions, cuts, velocity wounds (e.g., gunshot wounds), penetration wounds, puncture wounds, contusions, hematomas, tearing wounds, and/or crushing injuries. In one aspect, the term “wound” refers to an injury to the skin and subcutaneous tissue initiated in any one of a variety of ways (e.g., pressure sores from extended bed rest, wounds induced by trauma, cuts, ulcers, burns and the like) and with varying characteristics. Skin wounds are typically classified into one of four grades depending on the depth of the wound: (i) Grade I: wounds limited to the epithelium; (ii) Grade II: wounds extending into the dermis; (iii) Grade III: wounds extending into the subcutaneous tissue; and (iv) Grade IV (or full-thickness wounds): wounds wherein bones are exposed (e.g., a bony pressure point such as the greater trochanter or the sacrum).

As used herein, the term “wound healing” refers to a process by which the body of a wounded organism initiates repair of a tissue at the wound site (e.g., skin). The wound healing process requires, in part, angiogenesis and revascularization of the wounded tissue. Wound healing can be measured by assessing such parameters as contraction, area of the wound, percent closure, percent closure rate, and/or infiltration of blood vessels as known to those of skill in the art or as described herein in the section entitled “Wound healing assays”.

The term “subject” as used herein includes, without limitation, a human, mouse, rat, guinea pig, dog, cat, horse, cow, pig, monkey, chimpanzee, baboon, or rhesus. In one embodiment, the subject is a mammal. In another embodiment, the subject is a human.

As used herein, the term “pharmaceutical composition” refers to the active agent in combination with a pharmaceutically acceptable carrier of chemicals and compounds commonly used in the pharmaceutical industry. The term “pharmaceutically acceptable carrier” excludes tissue culture medium.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in cell biology and molecular biology can be found in “The Merck Manual of Diagnosis and Therapy”, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); The ELISA guidebook (Methods in molecular biology 149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology are also be found in Benjamin Lewin, Genes IX, published by Jones & Bartlett Publishing, 2007 (ISBN-13: 9780763740634); Kendrew et al. (eds.), Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIG. 1A shows MATRIGEL™ plug angiogenesis, quantified by flow cytometry. Relative fraction of CD31+/CD45− endothelial cells infiltrated into the plug. Endothelial cells are increased in Tie2-CYP2J2-Tr, Tie2-CYP2C8-Tr and sEH-null mice and decreased in Tie2-sEH-Tr mice relative to WT mice; N=6-8 plugs/group, *p<0.05 vs. WT.

FIG. 1B shows that wound healing is accelerated in Tie2-CYP2J2-Tr, Tie2-CYP2C8-Tr and sEH-null mice, and suppressed in Tie2-sEH-Tr mice relative to WT as quantified by wound area after 7 days (left and central panel). Time course of delayed wound healing in Tie2-sEH-Tr mice (right panel). N=8 wounds/group; *p<0.05 vs. WT. Scale bar, 1 cm.

FIG. 1C shows neonatal retinal vessel formation is increased in Tie2-CYP2C8-Tr mice relative to WT mice on postnatal day 5. N=7 pups/group; *p<0.05 vs. WT.

FIG. 1D shows that liver regeneration is accelerated in Tie2-CYP2C8-Tr mice on day 4 following partial hepatectomy. N=5 mice/group; *p<0.05 vs. WT. Scale bar, 1 cm.

FIG. 1E shows that systemic administration of 14,15-EET (15 μg/kg/day) via minipump stimulates liver regeneration on day 4 following partial hepatectomy compared to vehicle-treated mice. N=5 mice/group; *p<0.05 vs. vehicle. Scale bar, 1 cm.

FIG. 1F shows that endometriosis (ectopically implanted uterine tissue) is increased in Tie2-CYP2C8-Tr mice on day 6. N=5 mice/group; *p<0.05 vs. WT. Scale bar, 5 mm.

FIG. 1G shows that systemic administration of 14,15-EET (15 μg/kg/day) via minipump stimulated endometriosis on day 6. Note vascularity of lesions in 14,15-EET treated mice (arrows) compared to white, avascular lesions in vehicle-treated mice. N=5 mice/group; *p<0.05 vs. Control. Scale bar, 5 mm.

FIG. 2A shows that the expression of sEH, but not CYP2J and CYP2C, is down-regulated in tumor (TEC) vs. normal (NEC) endothelial cells and in tumor lysates from larger LLC tumors (>5 cm³) vs. smaller LLC tumors (<1 cm³) (left panel). sEH expression (dark staining) is also down-regulated in liver metastasis compared to normal adjacent liver (right panel). Control tissue=mouse liver.

FIG. 2B shows that the growth of B16F10 melanoma, T241 fibrosarcoma, and LLC primary tumors in Tie2-CYP2C8-Tr, Tie2-CYP2J2-Tr, sEH-null and WT mice. Insets show representative tumors on day 22 (B16F10 melanoma and T241 fibrosarcoma) or day 31 (B16F10 melanoma) post-injection. N=10-14 mice/group; *p<0.05 vs. WT. Scale bar, 1 cm.

FIG. 2C shows that the primary T241 fibrosarcoma tumor growth is inhibited in Tie2-sEH-Tr mice on day 28. N=6 mice/group; *p<0.05 vs. WT.

FIG. 2D shows the increase in plasma 14,15-EET and 11,12-EET in sEH-null mice (day 22 post-T241 fibrosarcoma injection) and plasma 14,15-EET in Tie2-CYP2C8-Tr mice (day 16 post-LLC injection) relative to WT as measured by LC-MS/MS. N=5 mice/group (sEH-null mice) and N=6-8 mice/group (Tie2-CYP2C8-Tr and WT mice); *p<0.05 vs. WT.

FIG. 2E shows that the systemic administration of. 14,15-EET (15 μg/kg/day) via minipump increases primary LLC tumor growth. N=6 mice/group; *p<0.05 vs. WT.

FIG. 2F shows that the corneal tumor angiogenesis induced by LLC is increased in Tie2-CYP2C8-Tr and sEH-null mice on day 13 post-injection relative to WT. Photos are representative of N=5 eyes/group.

FIG. 3A shows that spontaneous Lewis lung carcinoma (LLC) metastasis is increased in Tie2-CYP2C8-Tr and sEH-null mice relative to WT 10 days after primary tumor removal (LLC resection). Blue insets show representative lung metastasis in transgenic and WT mice. N=5 mice/group; *p<0.05 vs. WT. The experiment was performed three times with similar results. Scale bar, 1 cm.

FIG. 3B shows that spontaneous LLC metastasis is decreased in Tie2-sEH-Tr relative to WT 17 days after primary tumor removal (LLC resection). N=6 mice/group; *p<0.05 vs. WT.

FIG. 3C shows that primary LLC axillary lymph node metastasis occurs in Tie2-CYP2J2-Tr but not in WT mice by day 22 post-injection. Inset shows representative axillary lymph node metastases 22 days post-injection of LLC in Tie2-CYP2J2-Tr. N=6 mice/group; *p<0.05 vs. WT. Scale bar, 1 cm.

FIG. 3D shows that B16F10 melanoma metastasis to lung is increased in Tie2-CYP2C8-Tr mice relative to WT 18 days after tail vein injection. Insets show representative lung, liver, and abdominal metastasis in Tie2-CYP2C8-Tr mice. WT mice do not develop liver metastasis. N=6 mice/group; *p<0.05 vs. WT. Scale bar, 1 cm.

FIG. 3E shows that systemic administration of 14,15-EET (15 μg/kg/day) via minipump increases spontaneous LLC lung metastasis and distant femoral lymph node metastasis. N=10 mice/group; *p<0.05 vs. vehicle control.

FIG. 4A shows that systemic administration of a soluble epoxide hydrolase inhibitor (t-AUCB) stimulates primary LLC-GFP tumor growth. Inset shows representative tumors after 13 days of treatment. N=6 mice/group; *p<0.05 vs. control.

FIG. 4B shows that t-AUCB increases lung metastasis, and t-AUCB and TUPS increase liver metastasis in the spontaneous LLC metastasis model after 12 days of treatment. Vehicle-treated mice do not develop liver metastasis. Both t-AUCB and TUPS were given at dose of 10 mg/kg/day. N=8 mice/group; *p<0.05 vs. control.

FIG. 4C shows that t-AUCB and TUPS increase spontaneous B16F10 axillary lymph node metastasis after 21 days of treatment. t-AUCB and TUPS were given at dose of 10 mg/kg/day. N=6 mice/group; *p<0.05 vs. control.

FIG. 4D shows that TUPS increases liver regeneration at day 4 following partial hepatectomy. TUPS was given at dose of 10 mg/kg/day. N=5 mice/group; *p<0.05 vs. control.

FIG. 4E shows that the EET antagonist 14,15-EEZE (0.21 mg/mouse) inhibits primary LLC growth, prolongs survival and reduces plasma VEGF levels in a spontaneous LLC lung metastasis model. N=5 mice/group; *p<0.05 vs. control.

FIG. 4F shows that the EET antagonist 14,15-EEZE-mSI (0.21 mg/mouse) inhibits 14,15-EET- (15 μg/kg/day) induced spontaneous LLC metastasis. The stable EET metabolite 14,15-DHET (15 μg/kg/day) does not stimulate metastasis. N=5 mice/group; *p<0.05 vs vehicle control.

FIG. 5A shows that endothelial cell migration is decreased in Tie2-sEH-Tr mice but increased in Tie2-CYP2J2-Tr and Tie2-CYP2C8-Tr relative to WT (left panel). The sEH inhibitors t-AUCB and TUPS stimulate VEGF-mediated endothelial migration (middle panel). 14,15-EEZE inhibits VEGF-induced endothelial cell but not tumor cell (LLC) migration (right panel). N=3-4/group; *p<0.05 vs. WT or basal.

FIG. 5B shows that VEGF ELISA and western blot of plasma after heparin bead affinity purification show increased VEGF but not FGF2 in Tie2-CYP2C8-Tr and sEH-null mice 17 days post-B16F10 resection. N=5/group *p<0.05 vs. WT.

FIG. 5C shows that VEGF depletion with sFlt suppresses B16F10 tumor growth in Tie2-CYP2J2-Tr and sEH-null mice, but not in WT mice. N=5 mice/group; *p<0.05 vs. Ad-null control.

FIG. 5D shows that the sEH inhibitor t-AUCB does not promote spontaneous LLC metastasis in mice depleted of VEGF with sFlt (t-AUCB+sFlt). N=5 mice/group; *p<0.05 vs. t-AUCB alone.

FIG. 5E shows that the endogenous angiogenesis inhibitor TSP1 is down-regulated in plasma of Tie2-CYP2C8-Tr, sEH-null and Tie2-CYP2J2-Tr mice relative to WT on day 13 post-LLC injection.

FIG. 5F shows that the EET antagonist 14,15-EEZE (0.21 mg/mouse) does not significantly inhibit primary LLC tumor growth in TSP1 null mice. N=5 mice/group.

FIG. 5G shows that LLC tumors in VEGF-LacZ-Tr mice treated with 14,15-EET (15 μg/kg/day) show β-galactosidase staining (marker of VEGF production) in tumor endothelium and stromal fibroblasts (arrows). Scale bar, 20 μm.

FIG. 6A shows that endothelial cells isolated from Tie2-CYP2J2-Tr and Tie2-CYP2C8-Tr mice secrete significantly more 14,15-EET than cells isolated from WT mice. Endothelial cells isolated from Tie2-sEH-Tr mice secrete significantly less 14,15-EET than cells isolated from WT mice. N=3-4 per group; *p<0.05 vs. WT.

FIG. 6B shows that VEGF- but not FGF2-induced corneal angiogenesis is increased in Tie2-CYP2C8-Tr mice relative to WT. FGF2 (80 ng) induces no significant change in vessel length and neovascularization area in Tie2-CYP2C8-Tr vs. WT mice, whereas VEGF (160 ng) significantly stimulates vessel length and neovascularization area in Tie2-CYP2C8-Tr vs. WT mice. Neovascularization area is determined on day 6 by the formula 0.2×π×neovessel length×clock hours of neovessels. N=6 eyes per group; *p<0.05 vs. WT.

FIG. 6C shows that hematoxylin and eosin (H&E) stained sections of wounds in Tie2-CYP2J2-Tr and Tie2-CYP2C8-Tr mice on day 7 reveal a more mature wound with collagen deposition and minimal acute inflammation. Scale bar, 100 μm.

FIG. 6D shows that there are no significant changes in baseline liver weight/body weight ratio and liver weight on day 4 in Tie2-CYP2C8-Tr and WT mice after sham operation. Endothelial cell proliferation is increased in livers from Tie2-CYP2C8-Tr vs. WT mice on day 4 following partial hepatectomy as determined by immunofluorescent double staining for MECA-32 and Ki-67. Kidney regeneration is significantly increased in Tie2-CYP2C8-Tr mice 21 days after partial nephrectomy (right panel). N=5-8 mice/group; *p<0.05 vs. WT. Scale bar, 1 cm.

FIG. 6E shows that endometriosis is stimulated in Tie2-CYP2C8-Tr mice or by systemic administration of 14,15-EET (15 14/kg/day) as measured by percent of lesions established and area of established lesions. H&E staining of lesions shows endometrial glands in Tie2-CYP2C8-Tr mice on day 6. No endometrial glands are present in WT mice on day 6. Immunofluorescent double staining of lesion in Tie2-CYP2C8-Tr mice for endothelial cells (MECA-32) and proliferation (Ki-67) show prominent endothelial cell proliferation. N=5 mice/group; *p<0.05 vs. WT. Scale bar, 5 mm (in photo of lesion) and 20 μm (in immunofluorescent image).

FIG. 7A shows that sEH protein is down-regulated in tumor endothelial cells isolated from TRAMP mice compared to normal murine endothelial cells and to murine prostate tumor cells (TRAMP C1). The lower panel shows serial sections of B16F10 melanoma stained for CYP2J and CD31 and demonstrates that tumor endothelial cells express CYP2J. Scale bar, 20 μm.

FIG. 7B shows that CYP2J is localized to the endothelium of human hepatocellular carcinoma and human neuroblastoma. There is no staining with Rabbit IgG as a control. Scale bar, 20 μm.

FIG. 7C shows representative Tie2-sEH-Tr and WT mice with T241 fibrosarcoma tumors on day 25 post-injection.

FIG. 7D shows vessel density, as defined by the number of CD31-positive blood vessels, is increased in B16F10 melanoma in Tie2-CYP2C8-Tr, Tie2-CYP2J2-Tr, and sEH-null mice relative to WT mice on day 22 post-tumor implantation. The upper panel show photomicrographs (Scale bar, 20 μm) and the lower panel shows number of vessels per high power field. N=5 mice per group; *p<0.05 vs. WT.

FIG. 7E shows tumor angiogenesis as quantified by flow cytometry analysis of CD31+/CD45− endothelial cells in LLC on day 22 post-injection. Tumor ECs are increased 3-fold in Tie2-CYP2J2-Tr mice compared to WT mice. N=5 tumors/group; *p<0.05 vs. WT.

FIG. 8A shows that Tie2-CYP2C8-Tr and sEH-null mice exhibit liver and kidney metastasis (arrows) 10 days post LLC resection whereas WT mice do not. Representative photos are shown. Scale bar, 1 cm.

FIG. 8B shows that spontaneous LLC metastasis to lungs is decreased in Tie2-sEH-Tr vs. WT mice on day 17 post-LLC resection. Representative photos are shown. Scale bar, 1 cm.

FIG. 8C shows that Tie2-CYP2J2-Tr mice have increased LLC metastasis to the lung on day 22 post-LLC injection without resection. Left panel shows number of surface metastases and lung weight. Right panel shows representative photos. N=6 mice per group; *p<0.05 vs. WT. Scale bar, 1 cm.

FIG. 8D shows that H&E stained section of axillary lymph node metastasis 22 days post-injection of LLC in Tie2-CYP2J2-Tr mice reveals metastatic LLC tumor cells (arrows). Scale bar, 20 μm.

FIG. 8E shows in the upper panel that representative axillary lymph node metastasis 17 days post-B16F10 resection in WT, Tie2-CYP2C8-Tr and sEH-null mice. H&E stained sections of axillary lymph nodes confirm B16F10 tumor cell metastasis; Scale bar, 20 μm. The lower panel shows that the there is more than a 2-fold increase in lymph node metastasis vs. WT mice. N=6 mice/group; *p<0.05 vs. WT.

FIG. 8F shows that systemic administration of 14,15-EET (15 μg/kg/day) via minipump increases spontaneous lung and distant femoral lymph node metastasis 12 days post LLC resection. Representative photos are shown. N=10 mice/group. Scale bar, 1 cm.

FIG. 9A shows that analysis of plasma from LLC-GFP tumor bearing mice treated with the sEH inhibitor tAUCB (10 mg/kg/day) reveals an increase in plasma EETs by LC-MS/MS. N=5 mice per group; *p<0.05 vs. vehicle.

FIG. 9B shows that systemic administration of sEH inhibitors tAUCB and TUPS (10 mg/kg/day each) induces spontaneous liver metastasis 12 days post LLC resection and spontaneous axillary lymph node metastasis 21 days post B16F10 resection. Representative lungs and livers after 12 days of treatment and axillary lymph nodes after 21 days of treatment are shown. Scale bar, 1 cm.

FIG. 9C shows that systemic administration of the sEH inhibitor TUPS (10 mg/kg/day) accelerates wound healing relative to vehicle on day 4. Representative photo is shown. Scale bar, 1 cm.

FIG. 9D shows that the EET antagonist 14,15-EEZE-mSI inhibits lung metastasis induced by 14,15-EET. 14,15-DHET has no effect relative to control. Representative photographs on day 12 post LLC resection are shown. Scale bar, 1 cm.

FIG. 10A shows that Tie2-sEH-Tr mice exhibit endothelial-specific staining of sEH in the liver, whereas WT mice do not. Scale bar, 20 μm.

FIG. 10B shows that the sEH inhibitor tAUCB (10 mg/kg/day) is unable to promote primary LLC growth in mice depleted of VEGF by systemic sFlt. In contrast, primary LLC growth is promoted in tAUCB-treated mice receiving control virus. N=6 mice/group; *p<0.05 vs. tAUCB. Blue insets show representative photographs of LLC tumors. Scale bar, 1 cm.

FIG. 10C shows that 14,15-EET has no effect on in vitro tumor cell production of VEGF by LLC or B16F10. N=3/group.

FIG. 11A shows that cross-circulation between “EET high” and “EET low” mice is demonstrated after one animal in the pair was injected with 100 μl of 0.25% Evan's blue 4 weeks after surgical union. The first organ of the uninjected partner to show Evan's blue discoloration 30 minutes after injection is the liver. Scale bar, 1 cm.

FIG. 11B shows that three hours after injection, Evan's blue concentrations were equalized in various organs between the injected and uninjected partners. Spectrophotometric analysis of extravasated Evans blue is represented in bar graph (average±sem). N=5 mice/group.

FIG. 11C shows that the genotype of the tumor-bearing mouse (donor) determines growth of the primary tumor, regardless of the genotype of the recipient mouse. However, an EET-producing endothelium is critical at the metastatic site for EET-induced lung, liver and lymph node metastasis. Scale bar, 1 cm:

FIG. 11D shows that adoptive transfer of whole blood from the “low EET” recipient parabiont (Tie2-sEH-Tr), which exhibited no metastasis, into non-parabiosis “high EET” (Tie2-CYP2C8-Tr) mice caused metastatic disease and reduced survival. In contrast, adoptive transfer of whole blood from into WT mice did not cause metastatic disease and survival was 100%.

FIG. 12 shows the increase in lung regeneration in Tie2-CYP2C8 Tr mice and mice treated with TUPS following left pneumonectomy.

### DETAILED DESCRIPTION OF THE INVENTION

Embodiments of the methods described herein are based, in part, on the discoveries that modulating the levels of epoxyeicosatrienoic acids (EETs), lipid mediators produced by cytochrome P450 epoxygenases, can regulate tissue homeostasis and angiogenesis. Using genetic manipulation and pharmacological modulation of EET levels, the inventors found that EETs are critical for normal tissue growth, including angiogenesis, wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue) tissue regeneration, and organ regeneration.

Therefore, by modulating the level of EET, it is possible to affect angiogenesis in a tissue and some of the relate cellular events that are affected angiogenesis, for example, by increasing the level of EET, it is possible to promote or increase angiogenesis and promote cellular events such as cell proliferation, wound healing (encompassing but not limited to lacerations, abrasions, avulsions, cuts, velocity wounds, penetration wounds, puncture wounds, contusions, hematomas; tearing wounds, and/or crushing injuries to the skin and subcutaneous tissue) and organ/tissue regeneration. While not wishing to be bound by theory, the inventors showed that by inhibiting the degradation of EETs, the level of EETs in vivo is increased and this promoted increased angiogenesis, endothelial cell migration, and tissue/organ regeneration.

Accordingly, in one embodiment, provided herein is a method of wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue) and tissue and/or organ regeneration in a tissue in need thereof, the method comprising contacting the tissue with a therapeutically effective amount of a sEHi.

In yet another embodiment, provided herein is a method of promoting tissue growth or regeneration, the method comprising contacting the tissue with a therapeutically effective amount of a sEHi, whereby tissue growth or regeneration is enhanced relative to tissue growth or regeneration in the absence of the sEHi. In one embodiment, organ regeneration is specifically contemplated.

In one embodiment, provided herein is a method of promoting cell proliferation in a tissue in need thereof, the method comprising contacting the tissue with a therapeutically effective amount of a soluble epoxide hydrolase inhibitor (sEHi).

Four regioisomeric EETs (5,6-EET, 8,9-EET, 11,12-EET and 14,15-EET) have been investigated as autocrine and paracrine mediators of arachidonic acid-induced vasorelaxation in the cardiovascular and renal system. EETs play a role in tissue homeostasis which results from their effects on cellular proliferation, migration and inflammation (Spector, A. A. and Norris, A. W. Am J Physiol Cell Physiol 2007, 292, C996). Blood vessels also represent a major target of EETs (Spector, A. A. and Norris, A. W. Am J Physiol Cell Physiol 2007, 292, C996) which have been shown to stimulate angiogenesis (Pozzi, A. et al., J Biol Chem 2005, 280, 27138, Dunn, L. K. et al., Anat Rec A Discov Mol Cell Evol Biol 2005, 285, 771, Wang, Y. et al., J Pharmacol Exp Ther 2005, 314, 522). EETs are produced from arachidonic acid by cytochrome P450 (CYP) epoxygenases CYP2C8 and CYP2J2 and mainly metabolized by soluble epoxide hydrolase (sEH), also known as EPHX2, to less active dihydroxyeicosatrienoic acids (DHETs) (W. B. Campbell, W. B. and Falck, J. R. Hypertension 2007, 49, 590, Fleming, I. Trends Cardiovasc Med 2008, 18, 20) Inhibitors of sEH, which raise endogenous EET levels, are in clinical trials as anti-hypertensive agents (Imig, J. D. and Hammock, B. D., Nat Rev Drug Discov 2009, 8, 794).

Endogenously-produced lipid autacoids are locally-acting small molecule mediators that are known to play a central role in inflammation and in the response to tissue injury. These autacoids are best known as products of arachidonic acid metabolism by cyclooxygenases and lipoxygenases G. (Bannenberg, G. L. et al. Expert Opin Ther Pat 2009, 19, 663, Gronert, K. Mol Interv 2008 8, 28). Arachidonic acid is also a substrate for cytochrome P450 (CYP) epoxygenases CYP2C8 and CYP2J2, which convert it to four regioisomeric EETs (5,6-EET, 8,9-EET, 11,12-EET and 14,15-EET). The bioactive EETs are mainly metabolized by soluble epoxide hydrolase (sEH) to less active dihydroxyeicosatrienoic acids (DHETs) (W. B. Campbell, W. B. and Falck, J. R. Hypertension 2007, 49, 590, Fleming, I. Trends Cardiovasc Med 2008, 18, 20) EETs have been investigated as autocrine and paracrine mediators of arachidonic acid-induced vasorelaxation in the cardiovascular and renal system. CYP2C enzymes are induced by hypoxia, and it is believed that endothelial cells, which express CYPs, are a major source of EETs in the circulatory system during inflammation and angiogenesis (Fleming, I. Trends Cardiovasc Med 2008, 18, 20).

The enzyme soluble epoxide hydrolase (sEH) (EC3.3.2.10) catalyzes the reaction of a epoxide and water molecule to create a glycol molecule. The sEH belongs to the hydrolase family of enzymes, specifically those acting on ether bonds (ether hydrolases). Due to structural similarities, it has been proposed that the sEH evolved from the bacterial haloalkane dehalogenase. The systematic name of this enzyme class is epoxide hydrolase. Other names in common use include epoxide hydrase (ambiguous, epoxide hydratase (ambiguous), arene-oxide hydratase (ambiguous), aryl epoxide hydrase (ambiguous), trans-stilbene oxide hydrolase and cytosolic epoxide hydrolase. The human sEH, also known as epoxide hydrolase 2 (EPHX2) or cytosolic epoxide hydrolase (CEH), specifically catalyzes the conversion of epoxyeicosatrienoic acids (EpETrEs, EETs) to the corresponding dihydroxy eicosatrienoic acids (DiHETrEs, DHETs), thereby diminishing their vasodilator activity.

Inhibitors of sEH, which raise endogenous EET levels, are in clinical trials as anti-hypertensive agents (Imig, J. D. and Hammock, B. D., Nat Rev Drug Discov 2009, 8, 794). The role of EETs in tissue homeostasis results from their effects on cellular proliferation, migration and inflammation (Spector, A. A. and Norris, A. W. Am J Physiol Cell Physiol 2007, 292, C996). Blood vessels represent a major target of EETs (Spector, A. A. and Norris, A. W. Am J Physiol Cell Physiol 2007, 292, C996) which have been shown to stimulate angiogenesis (Pozzi, A. et al., J Biol Chem 2005, 280, 27138, Dunn, L. K. et al., Anat Rec A Discov Mol Cell Evol Biol 2005, 285, 771, Wang, Y. et al., J Pharmacol Exp Ther 2005, 314, 522).

In one embodiment, the tissue in need of cell proliferation, angiogenesis, wound healing, or tissue growth or regeneration is found in a subject.

In one embodiment, angiogenesis is enhanced or increased by the contacting.

In one embodiment, provided herein is a method of promoting cell proliferation in a subject in need thereof, the method comprising administering a therapeutically effective amount of a sEHi to the subject.

In another embodiment, provided herein is a method of wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue) or tissue and/or organ regeneration in a subject in need thereof, the method comprising administering a therapeutically effective amount of a sEHi to the subject.

In yet another embodiment, provided herein is a method of promoting tissue growth or regeneration in a subject in need thereof, the method comprising administering a therapeutically effective amount of a sEHi, whereby tissue growth or regeneration is enhanced relative to tissue growth or regeneration in the absence of the sEHi to the subject.

In one embodiment, the angiogenesis, cell proliferation, or tissue and/or organ growth or regeneration is enhanced by at least 5%. In other embodiments, the enhancement or increase is by at least 10%, at least 20%, at least 30%, at least 40%, at least 50%, at least 60%, at least 70%, at least 80%, at least 90%, or at least 100% or more.

In one embodiment, the sEHi inhibits of the activity of a soluble epoxide hydrolase (sEH) or inhibits the expression of a sEH gene in the tissue. For example, the sEHi is an antibody which can specifically bind to and inhibit sEH activity. For example, the sEHi inhibits the expression by RNA interference.

In one embodiment, the sEHi is selected from a group consisting of a small molecule, nucleic acid, nucleic acid analogue, protein, antibody, peptide, aptamer and variants or fragments thereof.

In one embodiment, the sEHi is a small molecule that inhibits the enzyme activity of sEH. In one embodiment, the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).

In one embodiment, the sEHi is one of the following compounds; entA-2b (Shen et al., Bioorg Med Chem Lett 2009 19:5314-20), AUDA (12-(3-adamantan-1-yl-ureido) dodecanoic acid) (Simpkins et al. Am J Pathol 2009 174:2086-95), compounds 27 and 28 as disclosed in Kasagami et al. Bioorg Med Chem Lett 2009, 19:1784-89, nbAUDA (the n-butyl ester of 12-(3-adamantan-1-yl-ureido)-dodecanoic acid) (Parrish et al Cell Biol Toxicol. 2009 25:217-25), compound 2 as disclosed in Morrisseau et al., Bioorg Med Chem Lett 2006 16:5439-5444, 4-(3-cyclohexylureido)-ethanoic acid (CU2), 4-(3-cyclohexylureido)-butyric acid (CU4), 4-(3-cyclohexylureido)-hexanoic acid (CU6), and 4-(3-cyclohexylureido)-heptanoic acid (CU7) (Gomez et al., Protein Sci 2006 15:58-64), 1-cyclohexyl-3-dodecylurea, 12-(3-cyclohexyl-ureido) dodecanoic acid and 950 [adamantan-1-yl-3-{5-[2-(2-ethoxyethoxy)ethoxy]pentyl}urea] (Olearczyk et al. J Pharmacol Exp Ther. 2006 318:1307-14), N-(1-Acetylpiperidin-4-yl)-N′-(adamant-1-yl)urea (5a) (Jones et al., Bioorg Med Chem Lett 2006 16:5212-6), 1,3-dicyclohexylurea (DCU), (Ghosh et al., Basic Clin Pharmacol Toxicol 2008 102:453-8, 1-adamantan-3-(5-(2-(2-ethylethoxy)ethoxy)pentyl)urea (AEPU) (Ulu et al., J Cardiovasc Pharmacol 2008 52:314-23), polyethylene glycol ester of AUDA, and 1-adamantan-1-yl-3-(5-(2-(2-ethoxyethoxy)ethoxy)-pentyl)urea as disclosed in Fife et al., J Pharmacol Exp Ther 2008 327:707-15, 12-(3-adamantan-1-yl-ureido)-dodecanoic acid butyl ester (AUDA-BE) (Motoki et al., Am J Physiol Heart Circ Physiol 2008 295:H2128-34, cis-4-[4-(3-adamantan-1-yl-ureido)-cyclohexyloxy]-benzoic acid (c-AUCB) (Jung et al., PLoS One 2010 5:e11979), 1-trifluoromethoxy-phenyl-3-(1-acetylpiperidin-4-yl)urea (TPAU) (Chiamnimonvat et al., J Cardiov Pharm 2007 50:225-237), and 1-(4-(4-(4-acetylpiperazin-1-yl)butoxy)phenyl)-3-adamantan-1-yl urea (Huang et al J Med Chem 2010 53: 8376-86).

In one embodiment, the sEHi is the compound AR9281 (1-(1-Acetyl-piperidin-4-yl)-3-adamantan-1-yl-urea), (Anandan et al., Bioorg Med Chem Lett 2011 21:983-8) used in the Phase II clinical trial “Evaluation of Soluble Epoxide Hydrolase (s-EH) Inhibitor in Patients with Mild to Moderate Hypertension and Impaired Glucose Tolerance” sponsored by Arete Therapeutics (US Government Clinical Trial ID: NCT00847899).

In one embodiment, the sEHi is the compound GSK2188931 (Kompa et al., European Heart Journal 2010 31:422 (Suppl.)) described in the clinical trial “Evaluation of the Effects of Urotensin-II and Soluble Epoxide Hydrolase Inhibitors on Skin Microvessel Tone in Patients With Heart Failure, and in Healthy Volunteers” sponsored by Monash University (US Government Clinical Trial ID: NCT00654966).

In other embodiments, the sEHi is selected from the inhibitors disclosed in the following U.S. patent applications: US 2010/0267807, US 2006/0293292, US 2010/0016310, US 2010/0074852, US 2006/0035869, US 2004/0092487, US 2007/0117782, US 2009/0215894, US 2008/0200444, US 2006/0276515, US 2009/0197916, US 2008/0221104, US 2009/0270382, US 2008/0200467, US 2009/0247521, US 2009/0270452, US 20009/0023731, US 2009/0099184, US 2008/0280904. In another embodiment, the sEHi is selected from the inhibitors disclosed in the following scientific publications: Morrisseau et al., Bioorg Med Chem Lett 2006 16:5439-5444; Morisseau, C., et al. Biochemical Pharmacology 2002, 63, 1599; Jones, P. D., et al. Bioorganic & medicinal chemistry letters 2006, 16, 5212, Wolf, N. M. et al. Analytical Biochemistry 2006, 355, 71, Morisseau, C. et al. Proceedings of the National Academy of Sciences 1999, 96, 8849, Xie, Y., et al. Bioorganic & Medicinal Chemistry Letters 2009, 19, 2354, Anandan, S., et al. Bioorganic & Medicinal Chemistry Letters, 2009, 19, 1066, Eldrup, A. B. et al. Bioorganic & Medicinal Chemistry Letters 2010, 20, 571, Taylor, S. J. et al. Bioorganic & Medicinal Chemistry Letters 2009, 19, 5864, Kasagami, T. et al. Bioorganic & Medicinal Chemistry Letters 2009, 19, 1784, Anandan, S, and Gless, R. D. Bioorganic & Medicinal Chemistry Letters 2010, 20, 2740, Shen, H. C. et al. Bioorganic & Medicinal Chemistry Letters 2009, 19, 3398, Shen et al., Bioorg Med Chem Lett 2009 19:5716-21, Kim, et al. Bioorg Med Chem Lett 2007, 15:312-23, Kim et al., J Med Chem 2004 47:2110-22, Marino Curr Top Med Chem 2009 9:452-63, Qiu et al., Cardiovasc Ther 2010 E-pub, PMID: 20433684, Shen Expert Opin Ther Pat 2610 20:941-56, and Eldrup, A. B. et al. Journal of Medicinal Chemistry 2009, 52, 5880.

In one embodiment the sEHi is a 1) pyrazole phenyl derived amide, 2) N-substituted pridinone or pyrimidine derivative, 3) acyl hydrazone, 4) Aniline-derived amide, 5) Compound 61, 6) Benzimidazole-5-carboxamide, or 7) 3,3 disubstituted piperidine-derived urea.

In one embodiment, the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited. Such sEHi can inhibit the transcription of the sEH gene or the translation of the sEH mRNA transcribed from the sEH gene.

In one embodiment, the methods described herein are applied in the context of promoting wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.

In one embodiment, the methods described herein comprise administering a sEH inhibitor (sEHi) to tissues in need of cell proliferation, angiogenesis, wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), or tissue growth or regeneration.

In one embodiment, the method of promoting angiogenesis in a tissue in need thereof includes but is not limited to tissues that require re-vascularization after disease and trauma. Re-vascularization is needed for the rehabilitation of important organs, such as the heart, liver, and lungs, after damage caused by disease and physical trauma (e.g., myocardial infarction, occlusive peripheral vascular disease). Diseases that halt, block or reduce blood circulation include, but are not limited to, stroke, heart attack, myocardial ischemia, ischemic limbs, diabetes, vascular diseases such as peripheral vascular disease (PVD), carotid artery disease, atherosclerosis, and renal artery disease. Trauma such as those from car accidents and shock can result in reduced blood circulation to areas needing increased circulation during the healing process. In addition, treatment of a subject with a sEH inhibitor described herein is applicable to improving wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), tissue and/or organ regeneration, collateral coronary, peripheral artery, and carotid circulation in patients suffering from impaired wound healing, neuropathy, impotence, erectile dysfunction, diabetic neuropathy, spinal cord injury, nerve injury, and other vascular occlusive disorders such as sickle cell disease, and stroke.

In one embodiment, the method of promoting angiogenesis is applied to erectile dysfunction, which can be caused by vascular disorders. The use of a sEH inhibitor described herein can treat impotence by encouraging repair of the penile vascular network.

In one embodiment, the method of promoting cell proliferation, promoting angiogenesis and/or tissue growth or regeneration is applied in the context of wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), tissue and/or organ repair and regeneration, fertility, erectile dysfunction, cardiac hypertrophy, tissue grafts, and/or tissue engineered constructs. A variety of tissues, or organs comprising organized tissues, requiring angiogenesis include but are not limited to the skin, muscle, gut, connective tissue, joints, bones and the like types of tissue in which blood vessels are required to nourish the tissue.

In one embodiment, the methods of promoting cell proliferation, angiogenesis, wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), or tissue and/or organ growth or regeneration, further comprise contacting a tissue with additional pro-angiogenic factors and/or growth promoting factors, e.g. VEGF, FGF, PDGF, and IGF. A number of biomolecules which induce or promote angiogenesis in tissues have been identified. The most prominent of these are: growth factors such as vascular endothelial growth factor (VEGF), fibroblast growth factor (FGF), epidermal growth factor (EGF), platelet-derived growth factors (PDGFs) and transforming growth factors (TGFs); and nitric oxide (NO). Therefore, in one embodiment, the method of promoting cell proliferation and/or promoting angiogenesis in a tissue-engineered construct further comprises administration of additional growth factors such as VEGF, FGF, EGF, PDGFs, TGFs, NO, and combinations thereof.

In one aspect, promoting cell proliferation, angiogenesis, wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), or tissue and/or organ growth or regeneration can protect severely hypertrophied hearts from ischemic injury. Myocardial hypertrophy is associated with progressive contractile dysfunction, increased vulnerability to ischemia-reperfusion injury, and is, therefore, a risk factor in cardiac surgery. During the progression of hypertrophy, a mismatch develops between the number of capillaries and cardiomyocytes (heart muscle cells) per unit area, indicating an increase in diffusion distance and the potential for limited supply of oxygen and nutrients. Treatment of hypertrophied hearts with VEGF resulted in an increase of microvascular density, improved tissue perfusion, and glucose delivery. (I. Friehs, et. al., 2004, The Annals of Thoracic Surgery, 77: 2004-2010). While not wishing to be bound by theory, the methods described herein for promoting cell proliferation and/or promoting angiogenesis can address this mismatch by potentiating the effect of VEGF in increasing the capillaries to improve the supply of nutrients to the cardiomyocytes.

In another aspect, promoting cell proliferation, angiogenesis, wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue), or tissue and/or organ growth or regeneration can stimulate bone repair and bone turnover. Several growth factors are known to be expressed in a temporal and spatial pattern during fracture repair. Exogenously added VEGF enhances blood vessel formation, ossification, and new bone maturation (Street, J. et. al., 2002, PNAS, 99:9656-61). Accordingly, the method described herein for promoting cell proliferation and/or promoting angiogenesis with a sEH inhibitor can be a therapy for bone repair.

In some aspects, the methods described herein for promoting cell proliferation, promoting angiogenesis and/or tissue growth or regeneration are applicable to the treatment of wounds, and particularly for the treatment of persistent wounds, such as diabetic ulcers. Wounds, in particular persistent wounds, which are difficult to heal, require a blood supply that can nourish the wound, mediate the healing process and minimize scar formation. Commonly used therapies for treating persistent wounds do not assist the wound to provide its own blood supply and therefore the healing process remains slow. Persistent wounds can be ischemic wounds, for example, where the injury results from lack of oxygen due to poor circulation such as in diabetes, scleroderma, and the like. Scleroderma is a disease involving an imbalance in tissue reformation giving rise to the overproduction of collagen, and ultimately resulting in swelling and hardening of the skin (and affected organs). Diabetic wounds are especially difficult to treat because the inadequate blood supply is often complicated by other medical conditions such as peripheral vascular disease and neuropathy.

SEH inhibitors can be used to promote wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue). A sEH inhibitor used for wound healing will promote more rapid wound closure and/or greater angiogenesis at a given time relative to a similar wound not treated with the sEH inhibitor. Wound healing assays are provided herein (see section entitled “Wound Healing Assays”) to test the wound healing activity of pharmaceutical compositions comprising the sEH inhibitors described herein.

In one embodiment, the sEHi is administered locally. For example, in a wound or bone fracture, the sEHi is applied directly to the wound or at bone fracture to speed healing. The sEHi can be administered when the wound is being dressed or when the fractured kale in being set and aligned surgically, e.g., with titanium plates and screws. When bone grafting is employed for fusing bones, e.g., spinal vertebrate fusion, the sEHi can be mixed with the bone grafting material/matrices and applied locally to the bone needing fusion. For organ or tissue re-section due to trauma or disease, e.g., liver, the sEHi can be applied directly to the organ or tissue to encourage organ or tissue regeneration.

In another embodiment, the sEHi can be applied in the form of a patch or scaffold material. In one embodiment, the patch facilitates sustain released of the inhibitor for a period of time. The patch or scaffold material can be applied locally, e.g., directly to the wound, bone fracture, organ and/or tissue.

In another embodiment, the sEHi administered intravenously to the wound, bone fracture, organ and/or tissue. For example, the sEHi is administered through the portal vein to the liver.

In one embodiment, the methods described herein comprise administering a sEH inhibitor topically to promote wound healing (encompassing but not limited to lacerations, abrasions, avulsions, cuts, velocity wounds, penetration wounds, puncture wounds, contusions, hematomas, tearing wounds, and/or crushing injuries to the skin and subcutaneous tissue). In one embodiment, the sEH inhibitor is incorporated into a hydrogel or dressing or the like for use in the treatment of wounds. Alternatively, the sEH inhibitor compositions can be administered systemically.

In some aspects, the methods described herein for promoting cell proliferation, promoting angiogenesis and/or tissue growth or regeneration can promote angiogenesis in 3-D scaffold constructs of biodegradable polymeric scaffolds coated with the sEH inhibitor. This equally applies to other scaffold materials (such as hydroxylapatite and metals). The emergence of the tissue engineering (TE) field has resulted in the development of various interdisciplinary strategies primarily aimed at meeting the need to replace organs and tissues lost due to diseases or trauma. In essence, the main TE approach is centered on seeding biodegradable scaffolds (both organic and inorganic such as poly(lactide-co-glycolide) and apatites) with donor cells, and optionally appropriate growth factor(s), followed by culturing and implantation of the scaffolds to induce and direct the growth of new, functional tissue. The scaffold material eventually disappears through biodegradation and is replaced by the specific tissue. This scaffold-guided TE approach is aimed at creating tissues such as skin, cartilage, bone, liver, heart, breast, etc.

Despite success with small (thin) tissue-engineered constructs, perhaps the biggest roadblock in scaffold-guided TE is engineering large tissue volumes. This challenge arises due to the lack of rapid vascularization (angiogenesis) of large three-dimensional (3-D) scaffold constructs. Accordingly, angiogenesis is a pre-requisite for scaffold-guided TE of large tissue volumes. Described herein is a method of promoting cell proliferation and/or promoting angiogenesis in a tissue-engineered construct, the method comprising contacting the tissue construct with a composition comprising a sEH inhibitor as that term is defined herein.

In some aspects, the methods described herein for promoting cell proliferation, promoting angiogenesis and/or tissue growth or regeneration are applicable to the regeneration of damage and underdeveloped organs or tissues. For example, the liver is damaged due to traumatic injury and the damaged portion is removed surgically. In the case of hepatic cancer, the cancerous lesions on the liver is also removed. In one embodiment, the sEHi can be administered directly to the liver during surgery to promote liver regeneration. In other embodiments, the sEHi can be administered systemically to the liver, e.g., via injection into a portal to the hepatic artery, after surgery to promote liver regeneration.

In pre-mature babies, the lungs are often underdeveloped. Embodiments of the methods described herein can be used to enhance development of such lungs. For example, sEHi can be administered systemically to pre-mature babies or administered as nebulizer inhalation forms. In one embodiment, the sEHi can be administered in conjunction with surfactants that are often administered to newborn to help with gaseous exchange in the underdeveloped lungs.

In one embodiment, provided herein is a composition comprising a pharmaceutically acceptable carrier and a sEH inhibitor.

In one embodiment, described herein is a method of promoting cell proliferation in a tissue in need thereof, the method comprising contacting the tissue with a composition comprising a sEH inhibitor.

In one embodiment, described herein is a method of promoting angiogenesis in a tissue in need thereof, the method comprising contacting the tissue with a composition comprising a sEH inhibitor.

The patient treated according to the various embodiments described herein is desirably a human patient, although it is to be understood that the principles of the invention indicate that the invention is effective with respect to all mammals, which are intended to be included in the term “patient”. In this context, a mammal is understood to include any mammalian species in which treatment of diseases associated with angiogenesis is desirable, particularly agricultural and domestic mammalian species.

In methods of treatment as described herein, the administration of sEH inhibitors can be for either “prophylactic” or “therapeutic” purpose. When provided prophylactically, the sEH inhibitor is provided in advance of any symptom. When provided therapeutically, a sEH inhibitor as described herein is provided at (or after) the onset of a symptom or indication of insufficient angiogenesis.

### Pro-Angiogenic Factors

Pro-angiogenic factors are factors that directly or indirectly promote new blood vessel formation. These factors can be expressed and secreted by normal and tumor cells. Pro-angiogenic factors comprising a sEHi as described can be administered in combination with other pro-angiogenic factors including, but not limited to, EGF, E-cadherin, VEGF (particularly VEGF isoforms: VEGF 121, 145 and 165), angiogenin, angiopoietin-1, fibroblast growth factors: acidic (aFGF) and basic (bFGF), fibrinogen, fibronectin, heparanase, hepatocyte growth factor (HGF), insulin-like growth factor-1 (IGF-1), IGF, BP-3, PDGF, VEGF-A VEGF-C, pigment epithelium-derived factor (PEDF), vitronection, leptin, trefoil peptides (TFFs), CYR61 (CCN1) and NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), and inflammatory cytokines and chemokines that are inducers of angiogenesis and increased vascularity, eg. CCL2 (MCP-1), interleukin-8 (IL-8) and CCL5 (RANTES).

### Angiogenesis Assays

Various methods of assaying for angiogenesis are described herein and referenced below. The complete content of these references is hereby incorporated by reference. In general, to measure the pro-angiogenic activity of an agent, e.g., a sEH inhibitor as described herein, one will perform a given assay in the presence and absence of the composition.

Examples of well described angiogenesis assays that can be used to test or confirm pro-angiogenic activity of the sEH inhibitors described herein include, but are not limited to in vitro endothelial cell assays, rat aortic ring angiogenesis assays, cornea micro pocket assays (corneal neovascularization assays), and chick embryo chorioallantoic membrane assays (Erwin, A. et al. (2001) Seminars in Oncology 28(6):570-576).

Some examples of in vitro endothelial cell assays include methods for monitoring endothelial cell proliferation, cell migration, or tube formation. It is anticipated that sEH inhibitors as described herein will affect each of these endothelial cell processes. Cell proliferation assays can use cell counting, BRdU incorporation, thymidine incorporation, or staining techniques (Montesano, R. (1992) Eur J Clin Invest 22:504-515; Montesano, R. (1986) Proc Natl. Acad. Sci USA 83:7297-7301; Holmgren L. et al. (1995) Nature Med 1:149-153).

As one example of a cell proliferation assay, human umbilical vein endothelial cells are cultured in Medium 199 (Gibco BRL) supplemented with 10% fetal bovine serum (Gibco BRL), 50 U/ml penicillin, 50 ng/ml streptomycin, 2 mM L-glutamine and 1 ng/ml basic fibroblast growth factor (bFGF) in T75 tissue culture flasks (Nunclon) in 5% CO₂ at 37° C. Cells are trypsinised (0.025% trypsin, 0.265 mM EDTA, GibcoBRL) and seeded in 96-well plates (Nunclon) at a density of 3000 cells/well/200 μl and cultured for 3 days. Cells are starved in 1% serum for 24 hours and are then treated with 1% serum containing 1 ng/ml bFGF in the presence or absence of a pro-angiogenic agent for a further 48 hours. Two hours before the termination of incubation, 20 μl of CELLTITER 96® Aqueous One Solution Reagent (Promega Inc.) is added info each well. After the completion of incubation at 37° C. in a humidified, 5% CO₂ atmosphere, the optical densities of the wells at 490 nm (“OD490”) are recorded using a plate reader (Bio-Tek). The quantity of formazan product as measured by the amount of 490 nm absorbance is directly proportional to the number of living cells in culture.

Alternatively, the incubation period of cells with the pro-angiogenic factor can be allowed to proceed for up to 7 days. The cells are counted on a coulter counter on e.g., days 1, 3, 5 and 7. Remaining cells are fed by media replacement on these days. Data is plotted and doubling time calculated using a regression analysis (cells in log phase of growth). The doubling time for the cell is monitored as an indicator of cell proliferative activity.

In cell migration assays, endothelial cells are plated on MATRIGEL™ and migration monitored upon addition of a chemoattractant (Homgren, L. et al. (1995) Nature Med 1:149-153; Albini, A. et al. (1987) Cancer Res. 47:3239-3245; Hu, G. et al. (1994) Proc Natl Acad Sci USA 6:12096-12100; Alessandri, G. et al. (1983) Cancer Res. 43:1790-1797.)

Another migration assay monitors the migration of bovine aortic endothelial cells. In the assay, bovine aortic endothelial (BAE) cells are allowed to grow to confluence in Dulbecco's modified Eagle medium (DMEM, GibcoBRL) containing 10% fetal bovine serum (GibcoBRL) in 12-well plates (Nunclon). The monolayers are then ‘wounded’ by scraping a disposable pipette tip across the dishes. After washing with Dulbecco's PBS plus calcium (0.1 g/L) (GIBCO™, Invitrogen Corporation), the wounded monolayers are cultured for a further 48 hours in fresh 1% serum in the presence or absence of a pro-angiogenic agent.

The degree of movement of cells in the wounded mono layers is determined by taking photomicrographs at the time of the initial wounding and 48 hours after wounding. The photomicrographs are taken at 20× magnification, e.g., on an Olympus CK2 inverted microscope and printed to a standard size of 15 cm wide by 10 cm deep. A grid with lines 1.5 cm apart and 10 cm long running parallel to a baseline is placed over the photograph. The baseline is placed on the “wounding line” above which the cells have originally been scraped off. The number of cells intercepted by each of the lines is recorded. This allows an assessment of the number of cells that have migrated 1.5, 3.0, 4.5, 6.0, 7.5 or 9.0 cm away from the baseline on the photomicrograph.

Endothelial tube formation assays monitor vessel formation (Kohn, E C. et al. (1995) Proc Natl Acad Sci USA 92:1307-1311; Schnaper, H W. et al. (1995) J Cell Physiol 165:107-118).

Rat aortic ring assays have been used successfully for the evaluation of angiogenesis drugs (Zhu, W H. et al. (2000) Lab Invest 80:545-555; Kruger, E A. et al. (2000) Invasion Metastas 18:209-218; Kruger, E A. et al. (2000) Biochem Biophys Res Commun 268:183-191; Bauer, K S. et al. (1998) Biochem Pharmacol 55:1827-1834; Bauer, K S. et al. (2000) J Pharmacol Exp Ther 292:31-37; Berger, A C. et al. (2000) Microvasc Res 60:70-80). Briefly, the assay is an ex vivo model of explant rat aortic ring cultures in a three dimensional matrix. One can visually observe either the presence or absence of microvessel outgrowths. The human saphenous angiogenesis assay, another ex vivo assay, can also be used (Kruger, E A. et al. (2000) Biochem Biophys Res Commun 268:183-191).

Another common angiogenesis assay is the corneal micropocket assay (Gimbrone, M A. et al. (1974) J Natl Cane Inst. 52:413-427; Kenyon, B M. et al. (1996) Invest Opthalmol V is Sci 37:1625-1632; Kenyon, B M. et al. (1997) Exp Eye Res 64:971-978; Proia, A D. et al. (1993) Exp Eye Res 57:693-698). Briefly, neovascularization into an avascular space is monitored in vivo. This assay is commonly performed in rabbit, rat, or mouse.

The chick embryo chorioallantoic membrane assay has been used often to study tumor angiogenesis, angiogenic factors, and antiangiogenic compounds (Knighton, D. et al. (1977) Br J Cancer 35:347-356; Auerbach, R. et al. (1974) Dev Biol 41:391-394; Ausprunk, D H. et al. (1974) Dev Biol 38:237-248; Nguyen, M. et al. (1994) Microvasc Res 47:31-40). This assay uses fertilized eggs and monitors the formation of primitive blood vessels that form in the allantois, an extra-embryonic membrane. This assay functions as an in vivo endothelial cell proliferation assay.

Other in vivo angiogenesis assays are described in U.S. Pat. No. 5,382,514 and the directed in vivo angiogenesis assay (DIVAA™) system made by Trevigen, Inc. In these assays, a pro-angiogenic factor is incorporated into a tissue compatible matrix or hydrogel material such as MATRIGEL™ (GibcoBDL) or in the angioreactor Cultrex® DIVAA™, the matrix material or angioreactor is implanted subdermally into nude mice. Over time, usually days, microvessels invade the matrix material or angioreactor. The matrix material or angioreactor are then excised from the host mouse and examined.

### Wound Healing Assays

The methods of administering sEH inhibitors described herein can be used to facilitate, enhance or accelerate wound healing. Wound healing, or wound repair, is an intricate process in which the skin (or some other organ) repairs itself after injury. The classic model of wound healing is divided into four sequential, yet overlapping, phases: (1) hemostasis, (2) inflammatory, (3) proliferative and (4) remodeling. Angiogenesis occurs during the proliferative phase of wound healing and promotes wound contraction (i.e., a decrease in the size of the wound). Microvascular in-growth into damaged tissue is an essential component of the normal healing process. In fact, wound therapy is often aimed at promoting neovascularization.

Thus, a wound healing assay can be used as an angiogenesis assay to assess the effect of a given sEHi described herein. Such wound healing assays include, but are not limited to, ear punch assays and full thickness dorsal skin assays. Wound healing assays can be performed as described in U.S. Published Application No. 20060147415, entitled “Composition and method for treating occlusive vascular diseases, nerve regeneration and wound healing,” which is incorporated herein by reference in its entirety. The term “full thickness” is used herein to describe a wound that includes the epidermal layer and at least a portion of the dermal layer. The term “full thickness” also encompasses a deep wound to the level of the panniculus carnosus that removes epidermal, dermal, subcutaneous, and fascia layers.

Full thickness dorsal skin wounding assays can be performed as described in e.g., Luckett-Chastain, L R and Galluci, R M, Br J Dermatol. (2009) Apr. 29; Shaterian, A et al., Burns (2009) May 5; Lee, W R, et al., Wound Repair Regen (2009) Jun. 12; and Safer, J D, et al., Endocrinology (2005) 146(10):4425-30, which are herein incorporated by reference in their entirety. Dorsal skin wounding assays can be performed using rat or mouse models.

Whereas, the ear punch wound assay is used to generate the wound healing data described herein, it is expected that any of the other wound healing assays described herein will provide similar or superior results with sEH inhibitors as described. In one embodiment, a full-thickness wound is effected by removing a section of skin (e.g., 1.5 mm diameter) from the dorsal surface (e.g., back) of an anesthetized animal by e.g., surgical incision. If so desired, the section of skin to be wounded can be pre-treated with a candidate pro-angiogenic factor prior to wound induction by e.g., subcutaneous injection. Alternatively, the wound can be treated using a candidate pro-angiogenic factor coincident with or immediately following wounding using methods known to one of skill in the art. The size, area, rate of healing, contraction and histology of the wound are assessed at different time points by methods known to those of skill in the art. The wound size of an animal is assessed by measuring the unclosed wound area compared to the original wound area. Wound healing can be expressed as either percent wound closure or percent wound closure rate. Wounds can be harvested at different time points by euthanizing the animal and removing a section of skin surrounding the wound site for histological analysis if so desired.

The capacity of a candidate pro-angiogenic factor to induce or accelerate a healing process of a skin wound can be determined by administering the candidate pro-angiogenic factor to skin cells colonizing the damaged skin or skin wound area and evaluating the treated damaged skin or wounds for e.g., angiogenesis and/or epidermal closure and/or wound contraction. As known to those of skill in the art, different administration methods (e.g., injection or topical administration) can be used to treat the skin wound, and different concentrations of the candidate pro-angiogenic factor can be tested. A statistically significant increase in the incidence of vessel formation and/or epidermal closure and/or wound contraction, over an untreated control, indicates that a tested candidate pro-angiogenic factor is capable of inducing or accelerating a healing process of a damaged skin or skin wound. Positive results are indicated by a reduction in the percent wound area of a mouse treated with a candidate pro-angiogenic factor of at least 5% compared to the wound area of an untreated or vehicle treated mouse at the same timepoint; preferably the reduction in percent wound area is at least 7%, at least 8%, at least 9%, at least 10%, at least 12%, at least 15%, at least 20%, at least 25%, at least 30%, at least 35%, at least 40%, at least 50%, at least 60%, at least 70%, at least 80%, at least 90%, at least 95%, at least 99%, or even 100% (i.e., wound is completely closed).

An ear punch model can also be used to assess rates of angiogenesis or wound healing, in a design similar to that for the full thickness dorsal back skin assay. The model consists of wounding the ear of an animal using a circular punch of a standard size (e.g., 2.25 mm). The wound is treated daily with either a MATRIGEL™ vehicle or a MATRIGEL™ containing a candidate pro-angiogenic factor.

### Bone Repair Assays

Method 1: Each mouse is anesthetized with a ketamine/xylazine anesthetic and an incision is made over the anteromedial surface of the right tibial diaphysis. The muscle is blunt dissected to expose the periosteal surface and a 0.6 mm diameter penetrating hole is created in the medial cortex approximately 1 mm distal from the termination of the tibial tuberosity. Following surgery and/or treatment with the sEH inhibitors described herein, all animals will undergo high resolution micro-CT scan (Scanco vivaCT 40; 11 μm voxel resolution) to confirm the fracture. A second and third micro-CT scan is performed in all animals at 12 and 21 days, respectively to monitor the progress of quantitative analysis of the bone mineral density at the fracture site.

Method 2: Each mouse is anesthetized with a ketamine/xylazine anesthetic and a small incision is made on the dorsolateral side of the thigh and was extended over the knee region. A longitudinal incision is made in the patellar tendon, and a 0.5 mm hole is drilled above the tibia tuberosity. A fracture is then made by cutting the shaft of tibia. A fracture generated in this manner is known to heal through both endochondral and intramembranous ossification.

sEHi described herein are mixed with MATRIGEL™ and injected into the fracture site using a microsyringe. The animals were allowed free, unrestricted weight bearing in cages after recovery from anesthesia. At different time points (3, 4, 7, 14, and 21 d) after the fracture, is analysed for the bone mineral density at the fracture site using a Small Animal Bone Densitometer.

Calvarial critical size bone defects assay. A critical size defect (˜5-mm diameter) in a rat calvaria is first created and the rats are locally treated with saline (control) or sEHi described herein for 28 days (100 μg/mice/5 days). After 28 days, analysis of bone regeneration can be determined by soft x-ray. The edges of the sEHi-treated calvaria would be expected to have a smaller aperture which indicates increase repair compared to those of the control animal treated with saline.

### Inhibitory Antibodies

In one embodiment, sEH antagonists are inhibitory antibodies against the enzyme activity of sEH. Such inhibitory antibodies can act by sterically hindering sEH interacting with EET substrate. Commercially available antibodies including mouse IgG monoclonal antibody against the human sEH (anti-sEH) include sEH Polyclonal Antibody (Cat #10010146) from Cayman Chemical, sEH antibodies A-5 (Cat #sc-166961), D-13 (Cat #sc-87099), F-17 (Cat # sc-22344), H-215 (Cat #sc-25797), and Y-13 9Cat #sc-87101) from Santa Cruz Biotechnology, Inc. USA, EPHX2 antibody (ab67788) from abcam, EPHX2 antibody (Cat #10833-1-AP) from Proteintech, and EPHX2 antibody (NBP1-02667) from Novus Biologicals. Alternatively, antibodies can be generated and synthesized by any methods known in the art and methods described herein.

The antibodies can be polyclonal or monoclonal antibodies. Antibodies are raised against the human sEH protein (SEQ. ID. No. 2; Genbank Accession No.: NP_{—}001970.2) or isoforms thereof (Genbank Accession No.: EAW63551, EAW63550, EAW63549, SAW63548, EAW63547). Alternatively, antibodies can be made by immunizing a mammal with an inoculum containing a recombinant DNA molecule that comprises a DNA sequence that contains a sequence encoding the human sEH. The recombinant DNA sequences are derived from the human sEH nucleic acid (Genbank Accession No.:NG_{—}012064.1) or mRNA of sEH (SEQ. ID. No.: 1, Genbank Accession No.:NM_{—}001979). Such method is described in U.S. Pat. No. 5,643,578 which is incorporated herein by reference in its entirety.

Methods for the production of antibodies against sEH are described in U.S. Pat. Nos. 6,072,037, 6,793,919, and WO 2007/070750 which are herein incorporated by reference in their entirety Inhibitory antibodies envisioned for the methods described herein include humanized antibodies, chimeric antibodies (e.g., an antibody with mouse variable region fused with human constant region), single chain antibodies, single-domain antibody, variant forms of humanized, chimeric or single chain antibodies that conserved amino acid substitutions at the non-antigen binding region such as in the immunoglobulin constant region (Fc), and any protein containing the antigen binding region of any inhibitory sEH antibody, including the Fab, F(ab)′2 or Fv fragment.

The inhibitory effect of the antibodies on sEH activity can be determined by testing the ability of the antibodies to inhibit sEH degeneration of EET. Such methods are well known in the art and can include, but are not limited to, a test for in vitro sEH activity in which the substrate (3-phenyl-oxiranyl)-acetic acid cyano-(6-methoxy-naphthalen-2-yl)-methyl ester (PHOME) is hydrolyzed by epoxide hydrolase into the fluorescent compound 6-methoxy-2-naphthaldehyde. Activity is determined by monitoring fluorescence using an excitation wavelength of 330 nm and emission wavelength of 465 nm (Cayman Chemical Cat #10011671). Multiple other assays are known in the art (see Morisseau and Hammock Current Protocols in Toxicology Vol. 33, 4.23.1-4.23.18, 2007, United States Patent Application 2010/0311775). These references are incorporated herein by reference in their entirety.

Antibodies for use in the methods described herein can be produced using any standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

The design of humanized immunoglobulins can be carried out as follows. When an amino acid falls under the following category, the framework amino acid of a human immunoglobulin to be used (acceptor immunoglobulin) is replaced by a framework amino acid from a CDR-providing non-human immunoglobulin (donor immunoglobulin): (a) the amino acid in the human framework region of the acceptor immunoglobulin is unusual for human immunoglobulins at that position, whereas the corresponding amino acid in the donor immunoglobulin is typical for human immunoglobulins in that position; (b) the position of the amino acid is immediately adjacent to one of the CDRs; or (c) the amino acid is capable of interacting with the CDRs (see, Queen et al. WO 92/11018, and Co et al., Proc. Natl. Acad. Sci. USA 88, 2869 (1991), respectively, both of which are incorporated herein by reference). For a detailed description of the production of humanized immunoglobulins see, Queen et al. and Co et. al.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (Morrison et al., Proc. Natl. Acad. Sci. 81:851-855 (1984); Neuberger et al., Nature 312:604-608 (1984); Takeda et al.; Nature 314:452-454 (1985)) by splicing genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies.

The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., Science 242:1038-1041 (1988)).

Methods for the production of antibodies are disclosed in PCT publication WO 97/40072 or U.S. Application. No. 2002/0182702, which are herein incorporated by reference. The processes of immunization to elicit antibody production in a mammal, the generation of hybridomas to produce monoclonal antibodies, and the purification of antibodies may be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entirety.

Once expressed, the whole antibodies, their dimers, individual light and heavy chains, or other immunoglobulin forms of the present invention can be purified according to standard procedures in the art, including ammonium sulfate precipitation, affinity columns, column chromatography, gel electrophoresis and the likes (see, generally, Scopes, R., Protein Purification, Springer-Verlag, N.Y. (1982), which is incorporated herein by reference in its entirety). Substantially pure immunoglobulins of at least about 90 to 95% homogeneity are preferred, and 98 to 99% or more homogeneity most preferred, for pharmaceutical uses.

### Nucleic Acid Inhibitors of sEH

In some embodiments, sEH inhibitors that inhibit the expression of a sEH are nucleic acids. Nucleic acid inhibitors of a sEH gene include, but not are limited to, RNA interference-inducing molecules (RNAi), for example, but not limited to, siRNA, dsRNA, stRNA, shRNA, an anti-sense oligonucleotide and modified versions thereof, where the RNA interference molecule silences the gene expression of the sEH gene. In some embodiments, the nucleic acid inhibitor of a sEH gene is an anti-sense oligonucleic acid, or a nucleic acid analogue, for example, but not limited to DNA, RNA, peptide-nucleic acid (PNA), pseudo-complementary PNA (pc-PNA), or locked nucleic acid (LNA) and the like. In alternative embodiments, the nucleic acid is DNA or RNA, or nucleic acid analogues, for example, PNA, pcPNA and LNA. A nucleic acid can be single or double stranded, and can be selected from a group comprising nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. Additional sequences can also be present.

RNA interference (RNAi) is a phenomenon in which double-stranded RNA (dsRNA) specifically suppresses the expression of a gene with its complementary sequence. Small interfering dsRNAs (siRNA) mediate post-transcriptional gene-silencing, and can be used to induce RNAi in mammalian cells. The dsRNA is processed intracellularly to release a short single stranded nucleic acid that can complementary base pair with the gene's primary transcript or mRNA. The resultant a double stranded RNA is susceptible to RNA degradation. Protein translation is thus prevent.

In some embodiments, single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

Protein expression from the sEH gene identified in SEQ. ID. No: 2 can be reduced by inhibition of the expression of polypeptide (e.g., transcription, translation, post-translational processing) or by “gene silencing” methods commonly known by persons of ordinary skill in the art.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76:9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be of at least 5%, 10%, 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

Double-stranded RNA (dsRNA) has been shown to trigger one of these posttranscriptional surveillance processes, in which gene silencing involves the degradation of single-stranded RNA (ssRNA) targets complementary to the dsRNA trigger (Fire A, 1999, Trends Genet 15:358-363). RNA interference (RNAi) effects triggered by dsRNA have been demonstrated in a number of organisms including plants, protozoa, nematodes, and insects (Cogoni C. and Macino G, 2000, Curr Opin Genet Dev 10:638-643).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA Apr.; 9(4):493-501, incorporated by reference herein in its entirety).

An siRNA can be substantially homologous to the sEH gene or genomic sequence, or a fragment thereof. As used in this context, the term “homologous” is defined as being substantially identical, sufficiently complementary, or similar to the sEH mRNA, or a fragment thereof, to effect RNA interference of the sEH gene. In addition to native RNA molecules, RNAs suitable for inhibiting or interfering with the expression of sEH gene include RNA derivatives and analogs. Preferably, the siRNA is identical to sEH mRNA.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides molecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The more preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

Locked nucleic acids (LNAs), also known as bridged nucleic acids (BNAs), developed by Wengel and co-workers (Koshkin A. A., 1998, Tetrahedron, 54:3607-3630) and Imanishi and co-workers (Obika S., 1998, Tetrahedron Lett., 39:5401-5404). LNA bases are ribonucleotide analogs containing a methylene linkage between the 2′ oxygen and the 4′ carbon of the ribose ring. The constraint on the sugar moiety results in a locked 3′-endo conformation that preorganizes the base for hybridization and increases melting temperature (Tm) values as much as 10° C. per base (Wengel J., 1999, Acc. Chem. Res., 32:301-310; Braasch D. A. and Corey, D. R., 2001, Chem. Biol., 8:1-7). LNA bases can be incorporated into oligonucleotides using standard protocols for DNA synthesis. This commonality facilitates the rapid synthesis of chimeric oligonucleotides that contain both DNA and. LNA bases and allows chimeric oligomers to be tailored for their binding affinity and ability to activate RNase H. Because oligomers that contain LNA bases have a native phosphate backbone they are readily soluble in water. Introduction of LNA bases also confers resistance to nucleases when incorporated at the 5′ and 3′ ends of oligomers (Crinelli R., et. al., 2002, Nucleic Acids Res., 30:2435-2443). The ability to use LNAs for in vivo applications is also favored by the finding that LNAs have demonstrated low toxicity when delivered intravenously to animals (Wahlestedt C., et. al., 2000, Proc. Natl. Acad. Sci. USA, 97: 5633-5638).

LNAs and LNA-DNA chimeras have been shown to be potent inhibitors of human telomerase and that a relatively short eight base LNA is a 1000-fold more potent agent than an analogous peptide nucleic acid (PNA) oligomer (Elayadi A. N., et. al., 2002, Biochemistry, 41: 9973-9981). LNAs and LNA-DNA chimeras have also been shown to be useful agents for antisense gene inhibition. Wengel and co-workers have used LNAs to inhibit gene expression in mice (Wahlestedt C., et. al., 2000, Proc. Natl. Acad. Sci. USA, 97:5633-5638), while Erdmann and colleagues have described the design of LNA-containing oligomers that recruit RNase H and have described the rules governing RNase H activation by LNA-DNA chimeras in cell-free systems (Kurreck J., et. al., 2002, Nucleic Acids Res., 30:1911-1918).

The syntheses of LNA-containing oligomers are known in the art, for examples, those described in U.S. Pat. Nos. 6,316,198, 6,670,461, 6,794,499, 6,977,295, 6,998,484, 7,053,195, and U.S. Patent Publication No. US 2004/0014959, and all of which are hereby incorporated by reference in their entirety.

Another nucleic acid derivative envisioned in the methods described herein is phosphorodiamidate morpholino oligomer (PMO). PMOs are DNA mimics that inhibit expression of specific mRNA in eukaryotic cells (Arora, V., et. al., 2000, J. Pharmacol. Exp. Ther. 292:921-928; Qin, G., et. al., 2000, Antisense Nucleic Acid Drug Dev. 10:11-16; Summerton, J., et. al., 1997, Antisense Nucleic Acid Drug Dev. 7:63-70). They are synthesized by using the four natural bases, with a base sequence that is complementary (antisense) to a region of a specific mRNA. They are different than DNA in the chemical structure that links the bases together. Ribose has been replaced with a morpholine group, and the phosphodiester is replaced with a phosphorodiamidate. These alterations make the antisense molecule resistant to nucleases (Hudziak, R., et. al., 1996 Antisense Nucleic Acid Drug Dev. 6:267-272) and free of charges at physiological pH, yet it retains the molecular architecture required for binding specifically to a complementary strand of nucleic acid (Stein, D., et. al, 1997, Antisense Nucleic Acid Drug Dev. 7:151-157; Summerton, J., et. al., 1997, Antisense Nucleic Acid Drug Dev. 7:63-70; Summerton, J., and D. Weller., 1997, Antisense Nucleic Acid Drug Dev. 7:187-195).

The synthesis, structures, and binding characteristics of morpholine oligomers are detailed in U.S. Pat. Nos. 5,698,685, 5,127,866, 5,142,047, 5,166,315, 5,521,063, and 5,506,337, and all of which are hereby incorporated by reference in their entirety. PMOs can be synthesized at AVI BioPharma (Corvallis, Oreg.) in accordance with known methods, as described, for example, in Summerton, J., and D. Weller U.S. Pat. No. 5,185,444; and Summerton, J., and D. Weller. 1997, Antisense Nucleic Acid Drug Dev. 7:187-195. For example, PMO against calcineurin or KCNN4 transcripts should containing between 12-40 nucleotide bases, and having a targeting sequence of at least 12 subunits complementary to the respective transcript. Methods of making and using PMO for the inhibition of gene expression in vivo are described in U.S. Patent Publication No. US 2003/0171335; US 2003/0224055; US 2005/0261249; US 2006/0148747; S 2007/0274957; US 2007/003776; and US 2007/0129323; and these are hereby incorporated by reference in their entirety.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting sEH can be readily designed and tested. Accordingly, siRNAs useful for the methods described herein include siRNA molecules of about 15 to about 40 or about 15 to about 28 nucleotides in length, which are homologous to sEH. Preferably, the siRNA molecules targeting sEH have a length of about 19 to about 25 nucleotides. More preferably, the siRNA molecules have a length of about 19, 20, 21, or 22 nucleotides. The siRNA molecules can also comprise a 3′ hydroxyl group. The siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment, the RNA molecule that targets sEH is double stranded—one strand has a 3′ overhang and the other strand can be blunt-ended or have an overhang. In the embodiment in which the sEH targeting RNA molecule is double stranded and both strands comprise an overhang, the length of the overhangs can be the same or different for each strand. In a embodiment, the RNA comprises at least 6, 7, 8, 9, 10, 11, 12, 13, 14, 15, 16, 17, 18, 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

In some embodiments, assessment of the expression and/or knock down of sEH using gene specific siRNAs can be determined by methods that are well known in the art, such as western blot analysis or enzyme activity assays. Other methods can be readily prepared by those of skill in the art based on the known sequence of the target mRNA.

siRNA sequences are chosen to maximize the uptake of the antisense (guide) strand of the siRNA into RISC and thereby maximize the ability of RISC to target the mRNA of sEH for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the mRNA of sEH.

In a preferred embodiment, the siRNA or modified siRNA is delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into a siRNA capable of targeting sEH. In one embodiment, the vector can be a plasmid, a cosmid, a phagmid, a hybrid thereof, or a virus. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

In some embodiments, an agent is protein or polypeptide or RNAi agent that inhibits the expression of sEH and/or activity of proteins encoded by sEH. In such embodiments, cells can be modified (e.g., by homologous recombination) to provide increased expression of such an agent, for example, by replacing, in whole or in part, the naturally occurring promoter with all or part of a heterologous promoter. so that the cells express the natural inhibitor agent. For example, a protein or miRNA inhibitor of sEH become expressed at higher levels. The heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of a peptide or nucleic acid agent inhibitor of sEH can also include an affinity column containing agents which will bind to the protein; one or more column steps over such affinity resins as concanavalin A-agarose, heparin-Toyopearl™ or Cibacrom blue 3GA Sepharose; one or more steps involving hydrophobic interaction chromatography using such resins as phenyl ether, butyl ether, or propyl ether; immunoaffinity chromatography, or complementary cDNA affinity chromatography.

In one embodiment, the nucleic acid inhibitors of sEH can be obtained synthetically, for example, by chemically synthesizing a nucleic acid by any method of synthesis known to the skilled artisan. The synthesized nucleic acid inhibitors of sEH can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability is desired, nucleic acids having nucleic acid analogs and/or modified internucleoside linkages can be preferred. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), dimethylene-sulfoxide (—CH2-SO—CH2), dimethylene-sulfone (—CH2-SO2-CH2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5,714,606 to Acevedo, et al, 5,378,825 to Cook, et al., 5,672,697 and 5,466,786 to Buhr, et al., 5,777,092 to Cook, et al., 5,602,240 to De Mesmacker, et al., 5,610,289 to Cook, et al. and 5,858,988 to Wang, also describe nucleic acid analogs for enhanced nuclease stability and cellular uptake.

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can also be chemically synthesized (Elbashir et al. (2001) Nature 411:494-98)

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi.

siRNA can also be produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. siRNA can be designed into short hairpin RNA (shRNA) for plasmid- or vector-based approaches for supplying siRNAs to cells to produce stable sEH silencing. Examples of vectors for shRNA are #AM5779: —pSilencer™ 4.1-CMV neo; #AM5777: —pSilencer™ 4.1-CMV hygro; #AM5775: —pSilencer™ 4.1-CMV puro; #AM7209: —pSilencer™ 2.0-U6; #AM7210: —pSilencer™ 3.0-H1; #AM5768: —pSilencer™ 3.1-H1 puro; #AM5762: —pSilencer™ 2.1-U6 puro; #AM5770: —pSilencer™ 3.1-H1 neo; #AM5764: —pSilencer™ 2.1-U6 neo; #AM5766: —pSilencer™ 3.1-H1 hygro; #AM5760: —pSilencer™ 2.1-U6 hygro; #AM7207: —pSilencer™ 1.0-U6 (circular) from Ambion®.

Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32). In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., the mRNA of sEH (SEQ. ID. NO.: 1), beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule of the present invention involves identifying the 23 nucleotide sequence motif AA(N19)TT (SEQ ID NO: 3) (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but are not limited to the NCBI, BLAST, Derwent and GenSeq as well as commercially available oligosynthesis software such as Oligoengine®, can also be used to select siRNA sequences against EST libraries to ensure that only one gene is targeted.

Methods of predicting and selecting antisense oligonucleotides and siRNA are known in the art and are also found at the GENSCRIPT, AMBION, DHARMACON, OLIGOENGINE websites and described in U.S. Pat. No. 6,060,248, which is hereby incorporated by reference in its entirety.

In some aspects, antisense nucleic acid technology can be used to inhibit the expression of the sEH gene. It is possible to synthesize a strand of nucleic acid (DNA, RNA or a chemical analogue) that will bind to the messenger RNA (mRNA) produced by that gene and inactivate it, effectively turning that gene “off”. This is because mRNA has to be single stranded for it to be translated. This synthesized nucleic acid is termed an “anti-sense” oligonucleotide because its base sequence is complementary to the gene's messenger RNA (mRNA), which is called the “sense” sequence (so that a sense segment of mRNA “5′-AAGGUC-3′” would be blocked by the anti-sense mRNA segment “3′-UUCCAG-5′”).

Delivery of RNA Interfering Agents: Methods of delivering RNA interfering agents, e.g., an siRNA, or vectors containing an RNA interfering agent, to the target cells (e.g., cells of a tissue needing angiogenesis), can include, for example (i) injection of a composition containing the RNA interfering agent, e.g., an siRNA, or (ii) directly contacting the cell, e.g. a cell in a tissue needing angiogenesis with a composition comprising an RNA interfering agent, e.g., an siRNA. In one embodiment, the RNA interfering agent can be targeted to a tissue expressing sEH. In another embodiment, RNA interfering agents, e.g., an siRNA can be injected directly into any blood vessel, such as vein, artery, venule or arteriole, via, e.g., hydrodynamic injection or catheterization. In yet another embodiment, the RNA interfering agent can be injected or applied topically directly to the site of a tissue in need of angiogenesis, regeneration, or wound healing.

Administration can be by a single injection or by two or more injections. The RNA interfering agent is delivered in a pharmaceutically acceptable carrier. One or more RNA interfering agents can be used simultaneously. The RNA interfering agents, e.g., the siRNAs targeting the mRNA of sEH, can be delivered singly, or in combination with other RNA interfering agents, e.g., siRNAs, such as, for example siRNAs directed to other cellular genes. siRNAs targeting sEH can also be administered in combination with other pharmaceutical agents which are used to treat or prevent immunological diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

RNA interfering agents, for e.g., an siRNA, can also be introduced into cells via the vascular or extravascular circulation, the blood or lymph system, and the cerebrospinal fluid.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

It is also known that RNAi molecules do not have to match perfectly to their target sequence. Preferably, however, the 5′ and middle part of the antisense (guide) strand of the siRNA is perfectly complementary to the sEH target nucleic acid sequence.

Accordingly, the RNAi molecules functioning as nucleic acid inhibitors of the sEH gene are, for example, but not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of that inhibit sEH expression do not include RNA molecules that comprise ssRNA greater than about 30-40 bases, about 40-50 bases, about 50 bases or more. In one embodiment, the siRNA molecules that inhibit sEH expression are double stranded for more than about 25%, more than about 50%, more than about 60%, more than about 70%, more than about 80%, more than about 90% of their length. In some embodiments, a nucleic acid inhibitor of a sEH gene is any agent which binds to and inhibits the expression of mRNA of sEH, where the mRNA or a product of transcription of nucleic acid is encoded by SEQ. ID NO: 1.

### Therapeutic/Prophylactic Administration

Pharmaceutical compositions administered according to the present invention can be applied, for example, topically to a tissue. The composition can be applied as a therapeutically effective amount in admixture with pharmaceutical carriers, in the form of topical pharmaceutical compositions. Such compositions include solutions, suspensions, lotions, gels, creams, ointments, emulsions, skin patches, etc. All of these dosage forms, along with methods for their preparation, are known in the pharmaceutical and cosmetic art. Harry's Cosmeticology (Chemical Publishing, 7th ed. 1982); Remington's Pharmaceutical Sciences (Mack Publishing Co., 18th ed. 1990). Typically, such topical formulations contain the active ingredient in a concentration range of 0.1 to 100 mg/ml, in admixture with a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to a sEH inhibitor with which it is admixed, unless so desired. The preparation of a pharmacological composition that contains active ingredients dissolved or dispersed therein is well understood in the art and need not be limited based on formulation. Other desirable ingredients for use in such preparations include preservatives, co-solvents, viscosity building agents, carriers, etc. The carrier itself or a component dissolved in the carrier may have palliative or therapeutic properties of its own, including moisturizing, cleansing, or anti-inflammatory/anti-itching properties. Penetration enhancers may, for example, be surface active agents; certain organic solvents, such as di-methylsulfoxide and other sulfoxides, dimethyl-acetamide and pyrrolidone; certain amides of heterocyclic amines, glycols (e.g. propylene glycol); propylene carbonate; oleic acid; alkyl amines and derivatives; various cationic, anionic, nonionic, and amphoteric surface active agents; and the like.

Topical administration of a pharmacologically effective amount can utilize transdermal delivery systems well known in the art. An example is a dermal patch.

In one embodiment, the pharmaceutical compositions described herein can be administered directly by injection, for example to the affected tissue, such as organ, muscle or tissue, or wound (encompassing but not limited to lacerations, abrasions, avulsions, cuts, velocity wounds, penetration wounds, puncture wounds, contusions, hematomas, tearing wounds, and/or crushing injuries to the skin and subcutaneous tissue). A preferred formulation is sterile saline or Lactated Ringer's solution. Lactated Ringer's solution is a solution that is isotonic with blood and intended for intravenous administration.

In a further embodiment, ophthalmic sEHi compositions are used to enhance functional recovery after damage to ocular tissues. Ophthalmic conditions that may be treated include, but are not limited to, retinopathies (including diabetic retinopathy and retrolental fibroplasia), macular degeneration, ocular ischemia, and glaucoma. Other conditions to be treated with the methods described herein include damage associated with injuries to ophthalmic tissues, such as ischemia reperfusion injuries, photochemical injuries, and injuries associated with ocular surgery. The ophthalmic compositions may also be used as an adjunct to ophthalmic surgery, such as by vitreal or subconjunctival injection following ophthalmic surgery. The sEHi compositions may be used for acute treatment of temporary conditions, or may be administered chronically, especially in the case of degenerative disease. The ophthalmic sEHi compositions may also be used prophylactically, especially prior to ocular surgery or noninvasive ophthalmic procedures or other types of surgery.

In one embodiment, the active compound is administered to a subject for an extended period of time to produce optimum wound healing, cell proliferation, or tissue regeneration. Sustained contact with the sEH inhibitor composition can be achieved by, for example, repeated administration of the sEH inhibitor composition over a period of time, such as one week, several weeks, one month or longer. More preferably, the pharmaceutically acceptable formulation used to administer the active compound provides sustained delivery, such as “slow release” of the active compound to a subject. For example, the formulation may deliver the active sEH inhibitor composition for at least one, two, three, or four weeks after the pharmaceutically acceptable formulation is administered to the subject.

As used herein, the term “sustained delivery” is intended to include continual delivery of the active sEH inhibitor composition in vivo over a period of time following administration, preferably at least several days, a week, several weeks, one month or longer. Sustained delivery of the active compound can be demonstrated by, for example, the continued therapeutic effect of the sEH inhibitor composition over time. Alternatively, sustained delivery of the sEH inhibitor composition may be demonstrated by detecting the presence of the sEH inhibitor composition in vivo over time.

Preferred approaches for sustained delivery include use of a polymeric capsule, a minipump to deliver the formulation, or a biodegradable implant. Implantable infusion pump systems (such as Infusaid; see such as Zierski, J. et al, 1988; Kanoff, R. B., 1994) and osmotic pumps (sold by Alza Corporation) are available in the art. Another mode of administration is via an implantable, externally programmable infusion pump. Suitable infusion pump systems and reservoir systems are also described in U.S. Pat. No. 5,368,562 by Blomquist and U.S. Pat. No. 4,731,058 by Doan, developed by Pharmacia Deltec Inc.

In addition to topical therapy it is contemplated that the pharmaceutical compositions described herein can also be administered systemically in a pharmaceutical formulation. Systemic routes include but are not limited to oral, parenteral, nasal inhalation, intratracheal, intrathecal, intracranial, and intrarectal. The pharmaceutical formulation is preferably a sterile saline or lactated Ringer's solution. For therapeutic applications, the preparations described herein are administered to a mammal, preferably a human, in a pharmaceutically acceptable dosage form, including those that may be administered to a human intravenously as a bolus or by continuous infusion over a period of time, by intramuscular, intraperitoneal, intracerebrospinal, subcutaneous, intra-arterial, intrasynovial, intrathecal, oral, or inhalation routes. For these uses, additional conventional pharmaceutical preparations such as tablets, granules, powders, capsules, and sprays may be preferentially required. In such formulations further conventional additives such as binding-agents, wetting agents, propellants, lubricants, and stabilizers may also be required.

The compositions can be formulated as a sustained release composition. For example, sustained-release means or delivery devices are known in the art and include, but are not limited to, sustained-release matrices such as biodegradable matrices or semi-permeable polymer matrices in the form of shaped articles, e.g., films, or microcapsules that comprise sEH inhibitors.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly(2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Other biodegradable polymers and their use are described, for example, in detail in Brem et al. (1991, J. Neurosurg. 74:441-446).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

Preferred micro particles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, osmotic mini pumps can be used to provide controlled sustained delivery of the pharmaceutical compositions described herein, through cannulae to the site of interest, e.g. directly into a tissue at the site of needing angiogenesis. The pump can be surgically implanted; for example, continuous administration of endostatin, an anti-angiogenesis agent, by intraperitoneally implanted osmotic pump is described in Cancer Res. 2001 Oct. 15; 61(20):7669-74. Therapeutic amounts of sEH inhibitors can also be continually administered by an external pump attached to an intravenous needle.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

For enteral administration, a composition can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet can be made by compression or molding, optionally with one or more accessory ingredients. Compressed tablets can be prepared by compressing in a suitable machine the active compound in a free-flowing form, e.g., a powder or granules, optionally mixed with accessory ingredients, e.g., binders, lubricants, inert diluents, surface active or dispersing agents. Molded tablets can be made by molding in a suitable machine, a mixture of the powdered active compound with any suitable carrier.

A syrup or suspension can be made by adding the active compound to a concentrated, aqueous solution of a sugar, e.g., sucrose, to which can also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for oral administration can be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS(N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

Formulations for rectal administration can be presented as a suppository with a conventional carrier, e.g., cocoa butter or Witepsol S55 (trademark of Dynamite Nobel Chemical, Germany), for a suppository base.

The route of administration, dosage form, and the effective amount vary according to the potency of the sEH inhibitor, its physicochemical characteristics, and according to the treatment location. The selection of proper dosage is well within the skill of an ordinarily skilled physician.

In one embodiment, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of compositions include polysaccharides such as sodium carboxymethylcellulose or methylcellulose, polyvinylpyrrolidone, polyacrylates, polyoxyethylene-polyoxypropylene-block polymers, polyethylene glycol and wood wax alcohols. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. No. 3,773,919, EP 58,481A, U.S. Pat. No. 3,887,699, EP 158,277A, Canadian Patent No. 1176565, U. Sidman et al., Biopolymers 22:547 (1983) and R. Langer et al., Chem. Tech. 12:98 (1982).

In one embodiment, other ingredients may be added to pharmaceutical formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the composition comprising sEHi described herein can include but are not limited to one or more bioactive agents to induce healing or regeneration of damaged tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increase mitosis for cell growth and cell differentiation.

A great number of growth factors and differentiation factors that are known in the art to stimulated cell growth and differentiation of the progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment, the pharmaceutical formulation to be used for therapeutic administration is sterile. Sterility is readily accomplished by filtration through sterile filtration membranes (e.g., 0.2 micron membranes).

For therapeutic applications, the appropriate dosage of compositions will depend upon the type of tissue needing angiogenesis or other beneficial effect of the sEH inhibitor, the associated medical conditions to be treated, the severity and course of the medical conditions, whether the compositions are administered for preventative or therapeutic purposes, previous therapy, the patient's clinical history and response to the compositions and the discretion of the attending physician. In addition, in vitro or in vivo assays can optionally be employed to help identify optimal dosage ranges. The precise dose to be employed will also depend on the route of administration, and the seriousness of the condition being treated and should be decided according to the judgment of the practitioner and each subject's circumstances in view of, e.g., published clinical studies. Suitable effective dosage amounts for topical administration of the sEH inhibitor compositions described herein range from about 10 micrograms to about 5 grams applied or administered about every 4 hours, although they are typically about 500 mg or less per every 4 hours. In one embodiment the effective dosage for topical administration is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered.

For systemic administration, the dosage ranges are typically from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

The compositions comprising an sEH inhibitor are suitably administered to the patient at one time or over a series of treatments. For purposes herein, a “therapeutically effective amount” of a composition comprising an sEH inhibitor is an amount that is effective to either prevent, reduce the likelihood, lessen the worsening of, alleviate, or cure one or more symptoms or indicia of the treated condition.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, a sEH inhibitor as described herein can be targeted to tissue- or tumor-specific targets by using bispecific antibodies, for example produced by chemical linkage of an anti-ligand antibody (Ab) and an Ab directed toward a specific target. The addition of an antibody to a sEH inhibitor permits the agent attached to accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

Some embodiments of the present invention can be defined as any of the following numbered paragraphs:
1. A method of promoting cell proliferation in a tissue in need thereof, the method comprising contacting the tissue with a therapeutically effective amount of a soluble epoxide hydrolase inhibitor (sEHi).
2. The method of paragraph 1, wherein angiogenesis is enhanced by the contacting.
3. The method of paragraph 1, wherein endothelial cell migration is enhanced by the contacting.
4. The method of paragraph 2, wherein the sEHi inhibits the activity of a soluble epoxide hydrolase (sEH) or inhibits the expression of a sEH gene in the tissue.
5. The method of paragraph 1, 2, 3, or 4 wherein the sEHi is selected from a group consisting of a small molecule, nucleic acid, nucleic acid analogue, protein, antibody, peptide, aptamer and variants or fragments thereof.
6. The method of any one of paragraph 1-5, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).
7. The method of any one of paragraph 1-5, wherein the sEHi is an antibody which can specifically bind to and inhibit sEH activity.
8. The method of any one of paragraph 1-5, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.
9. The method of paragraph 1-8, wherein the method is applied in the context of promoting wound healing, neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.
10. A method of promoting angiogenesis in a tissue in need thereof, the method comprising contacting the tissue with a therapeutically effective amount of a soluble epoxide hydrolase inhibitor (sEHi).
11. The method of paragraph 10, wherein the sEHi inhibits the activity of a soluble epoxide hydrolase (sEH) or inhibits the expression of a sEH gene in the tissue.
12. The method of paragraph 10 or 11, wherein the sEHi is selected from a group consisting of a small molecule, nucleic acid, nucleic acid analogue, protein, antibody, peptide, aptamer and variants or fragments thereof.
13. The method of any one of paragraphs 10-12, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).
14. The method of any one of paragraphs 10-12, wherein the sEHi is an antibody which can specifically bind to and inhibit sEH activity.
15. The method of any one of paragraphs 10-12, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.
16. The method of paragraphs 10-15, wherein the method is applied in the context of promoting wound healing, neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.
17. A method of promoting wound healing, the method comprising contacting a wound with a therapeutically effective amount of a soluble epoxide hydrolase inhibitor (sEHi), whereby wound healing is enhanced relative to wound healing in the absence of the sEHi.
18. The method of paragraph 17, wherein angiogenesis is enhanced by the contacting.
19. The method of paragraph 17 or 18, wherein the sEHi inhibits the activity of a soluble epoxide hydrolase (sEH) or inhibits the expression of a sEH gene in the tissue.
20. The method of paragraph 17, 18 or 19, wherein the sEHi is selected from a group consisting of a small molecule, nucleic acid, nucleic acid analogue, protein, antibody, peptide, aptamer and variants or fragments thereof.
21. The method of any one of paragraphs 17-20, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).
22. The method of any one of paragraphs 17-20, wherein the sEHi is an antibody which can specifically bind to and inhibit sEH activity.
23. The method of any one of paragraphs 17-20, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.
24. The method of any one of paragraphs 17-23, wherein the contacting comprises administration of a topical medicament comprising the sEHi.
25. The method of any one of paragraphs 1-24, wherein the contacting comprises administering a pharmaceutical composition comprising the sEHi and a pharmaceutically acceptable carrier to an individual.
26. Use of a soluble epoxide hydrolase inhibitor (sEHi) for promoting cell proliferation in a tissue in need thereof.
27. Use of a soluble epoxide hydrolase inhibitor (sEHi) for promoting wound healing in a tissue in need thereof.
28. Use of a soluble epoxide hydrolase inhibitor (sEHi) for promoting wound healing in a subject in need thereof.
29. Use of a soluble epoxide hydrolase inhibitor (sEHi) for the manufacture of medicament for promoting wound healing in a tissue in need thereof.
30. Use of a soluble epoxide hydrolase inhibitor (sEHi) for promoting tissue growth or regeneration in a subject in need thereof.
31. Use of a soluble epoxide hydrolase inhibitor (sEHi) for the manufacture of medicament for promoting tissue growth or regeneration in a subject in need thereof.
32. Use of any one of paragraphs 26-31, wherein angiogenesis is enhanced.
33. Use of any one of paragraphs 26-31, wherein the sEHi inhibits the activity of a soluble epoxide hydrolase (sEH) or inhibits the expression of a sEH gene in the tissue.
34. Use of any one of paragraphs 26-31, wherein the sEHi is selected from a group consisting of a small molecule, nucleic acid, nucleic acid analogue, protein, antibody, peptide, aptamer and variants or fragments thereof.
35. Use of any one of paragraphs 26-31, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).
36. Use of any one of paragraphs 26-31, wherein the sEHi is an antibody which can specifically bind to and inhibit sEH activity.
37. Use of any one of paragraphs 26-31, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.
38. A method of promoting tissue growth or regeneration, the method comprising contacting the tissue with a therapeutically effective amount of a soluble epoxide hydrolase inhibitor (sEHi), whereby tissue growth or regeneration is enhanced relative to tissue growth or regeneration in the absence of the sEHi.
39. A method of promoting tissue growth or regeneration in a subject, the method comprising administrating a therapeutically effective amount of a soluble epoxide hydrolase inhibitor (sEHi), whereby tissue growth or regeneration is enhanced relative to tissue growth or regeneration in the absence of administrating the sEHi.
40. The method of paragraph 38 or 39, wherein angiogenesis is enhanced by the contacting.
41. The method of any one of paragraphs 38-40, wherein the sEHi inhibits the activity of a soluble epoxide hydrolase (sEH) or inhibits the expression of a sEH gene in the tissue.
42. The method of any one of paragraphs 38-41, wherein the sEHi is selected from a group consisting of a small molecule, nucleic acid, nucleic acid analogue, protein, antibody, peptide, aptamer and variants or fragments thereof.
43. The method of any one of paragraphs 38-42, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea. (TUPS).
44. The method of any one of paragraphs 38-42, wherein the sEHi is an antibody which can specifically bind to and inhibit sEH activity.
45. The method of any one of paragraphs 38-42, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.
46. The method of any one of paragraphs 38-42, wherein the method is applied in the context of promoting wound healing, neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.

This invention is further illustrated by the following examples which should not be construed as limiting.

### EXAMPLES

### Example 1

### Endothelial-Derived EETs Regulate Tissue Growth

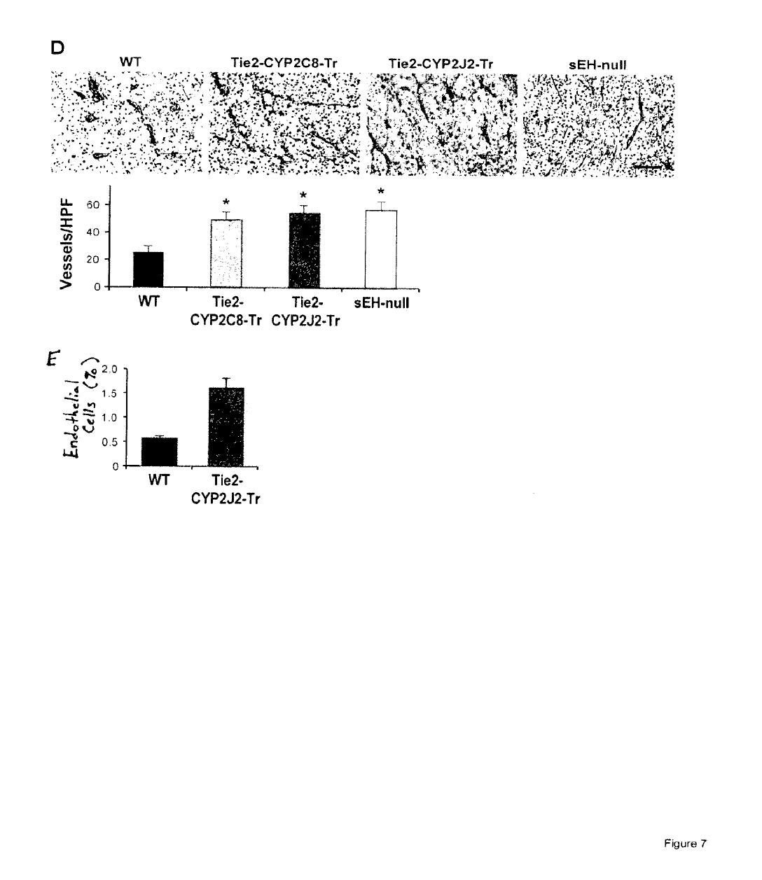

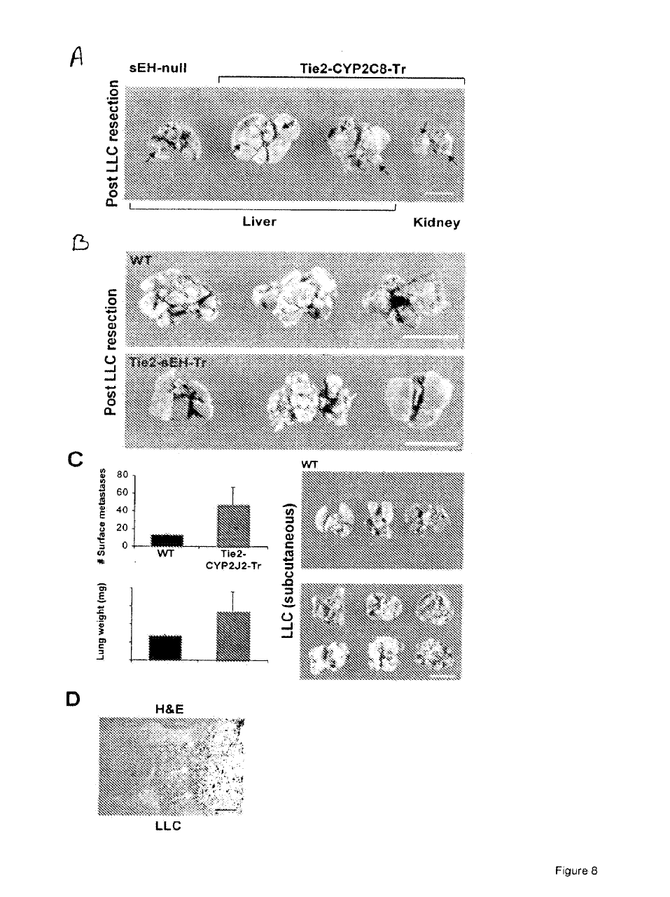

Three lines of transgenic mice with high endothelial EET levels were generated: mice with endothelial (Tie2-promoter driven) expression of either human CYP2C8 or human CYP2J2 (Tie2-CYP2C8-Tr, Tie2-CYP2J2-Tr) and mice with global disruption of the gene that encodes sEH (sEH-null) (C. J. Sinal et al., J Biol Chem 275, 40504, 2000). Tie2CYP2C8-Tr and Tie2-CYP2J2-Tr mice have significantly increased endothelial EETs compared to wild-type (WT) mice as measured by liquid chromatography-tandem mass spectrometry (LC/MS/MS) (FIG. 6A), and sEH-null mice have significantly increased plasma EETs (J. M. Seubert et al., Circ Res 99, 442 2006). In contrast, cyclooxygenase- and lipoxygenase-derived metabolites are unaffected in these mice. Mice with endothelial expression of human sEH (Tie2-sEH-Tr) that have significantly decreased endothelial EETs (FIG. 6A) were also generated. To determine whether these changes in endothelial-derived EETs affected physiologic or pathologic tissue growth in vivo, six well-characterized animal models were utilized: (i) angiogenesis MATRIGEL™ plug assay, (ii) corneal micropocket assay, (iii) wound healing, (iv) neonatal retinal vessel formation, (v) organ regeneration, and (vi) endometriosis.

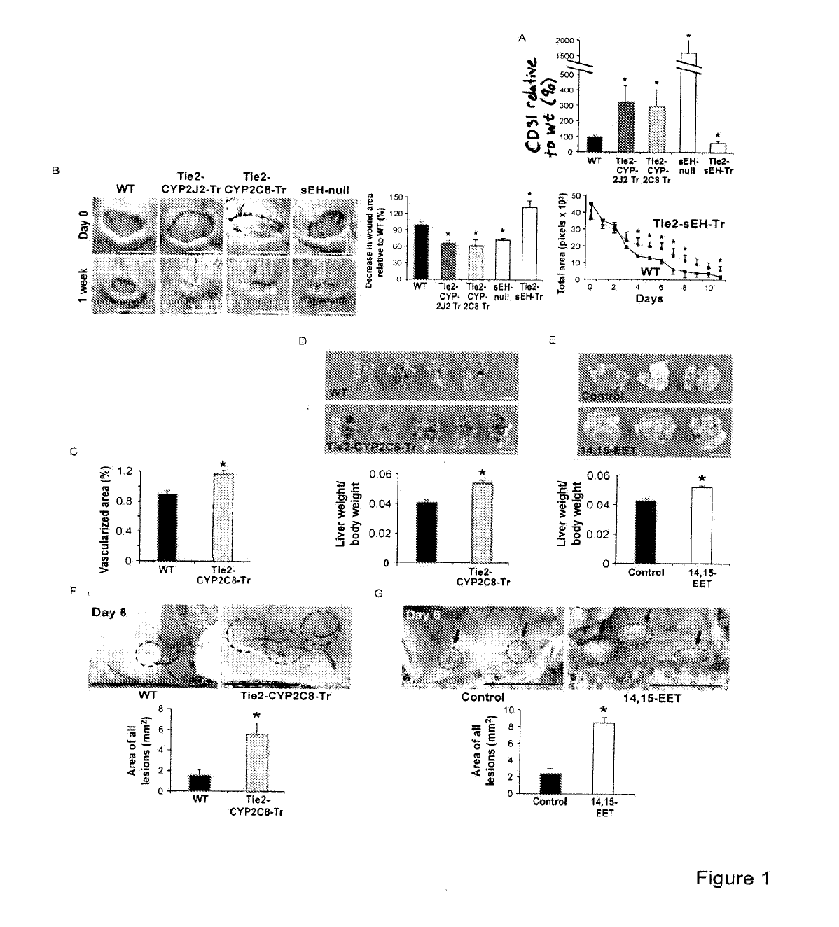

MATRIGEL™ plugs were implanted into mice in the absence of exogenous growth factors. Compared to WT mice, sEH-null, Tie2-CYP2J2-Tr and Tie2-CYP2C8-Tr mice each exhibited pronounced invasion of endothelial cells into the plug. This was indicated by an increase in CD31-positive microvessels when analyzed by whole-mount immunofluorescent staining (FIG. 1A), an observation which is consistent with the pro-angiogenic effects of EETs (Pozzi, A. et al., J Biol Chem 2005, 280, 27138, Dunn, L. K. et al., Anat Rec A Discov Mol Cell Evol Biol 2005, 285, 771, Wang, Y. et al., J Pharmacol Exp Ther 2005, 314, 522). Flow cytometry quantification of dissociated cells showed a 4-15 fold increase in CD31 positive cells in the plug (FIG. 1A). Conversely, in plugs from Tie2-sEH-Tr mice, there was a nearly 40% decrease in the number of infiltrating endothelial cells compared to WT mice (FIG. 1B). To determine whether the pro-angiogenic activity of endothelial-derived EETs is mediated by vascular endothelial growth factor (VEGF) (A. C. Webler et al., Am J Physiol Cell Physiol 295, C1292 2008, S. Yang, S. Wei, A. Pozzi, J. H. Capdevila, Arch Biochem Biophys 489, 82 2009) and/or fibroblast growth factor-2 (FGF2), corneal micro-pocket assays were performed in Tie2-CYP2C8-Tr and WT mice. Implantation of FGF2-(80 ng) or VEGF-(160 ng) containing pellets stimulated corneal neovascularization over 6 days in WT animals, as previously reported D. Panigrahy et al., J. Clin. Invest. 110, 923 2002). The area of neovascularization induced by FGF2 in Tie2-CYP2C8-Tr mice was unchanged relative to WT mice. In contrast, VEGF-stimulated angiogenesis in Tie2-CYP2C8-Tr mice was increased by approximately 60% when compared to WT mice, reflected by increased vessel length and neovascularization area (FIG. 6B).

Wound healing was accelerated in Tie2-CYP2J2-Tr, Tie2-CYP2C8-Tr, and sEH-null mice compared to WT mice one week after tissue injury (FIG. 1B). Immunohistochemical analysis revealed more mature wounds in the genetically altered mice with abundant collagen deposition, decreased inflammation and increased vascularization as measured by CD31 staining (data not shown). By contrast, wound healing in Tie2-sEH-Tr mice was suppressed (FIG. 1B). Analysis of the time-course revealed that the deficit in wound healing in the Tie2-sEH-Tr mice was due to a delay in the healing process rather than an inherent reduction of wound healing capacity (FIG. 1B). Consistent with this observation, the total area of newly formed microvessels in the neonatal retina, which is also tightly regulated by VEGF and its receptors (P. A. D'Amore, Invest Ophthalmol V is Sci 35, 3974 1994, A. Mammoto et al., Nature 457, 1103 2009), was significantly increased in Tie2-CYP2C8-Tr compared to WT mice on postnatal day 5 (FIG. 1C).

The influence of endothelial-derived EETs on liver regeneration, which has been shown to depend on VEGF-mediated angiogenesis (A. K. Greene et al., Ann Surg 237, 530 2003) was examined via a partial hepatectomy (removal of ⅔ of the liver). By day 4 following partial hepatectomy, Tie2-CYP2C8-Tr mice exhibited a 32% increase in the liver/body weight ratio when compared to WT controls (FIG. 1D). There were no significant differences in liver/body weight ratio at baseline or following sham operation (FIG. 6D). Histological analysis of the Tie2-CYP2C8-Tr livers revealed increased hepatocyte proliferation, multinucleation and increased nuclear size, features that are typical of dividing cells in a regenerating liver. Endothelial cell proliferation was increased in the Tie2-CYP2C8-Tr mice when compared to WT mice (FIG. 6D). Kidney regeneration was also significantly increased in Tie2-CYP2C8-Tr mice (FIG. 6D). To unambiguously demonstrate that this effect was specific to EETs and not to other downstream effects of CYP enzymes, and that it was sufficient to promote tissue regeneration, 14,15-EET was administered to mice by osmotic mini-pump. Consistent with the results in the Tie2-CYP2C8-Tr mice, systemic administration of 14,15-EET significantly increased the liver/body weight ratio by 21% compared with the vehicle (FIG. 1E).

Since endometrial implants behave like a malignancy with local and distant invasion and pronounced angiogenesis (M. A. Bedaiwy, M. A. Abdel-Aleem, A. Miketa, T. Falcone, Minerva Ginecol 61, 285 2009), endometriosis was also investigated. Endometriosis (total area and number of endometrial implants) was increased by 250% on day 6 in Tie2-CYP2C8-Tr mice when compared to WT mice (FIG. 1F). Increased vasculature and endothelial proliferation was evident in endometrial implants from Tie2-CYP2C8-Tr mice (FIG. 6E). Furthermore, systemic administration of 14,15-EET also increased endometriosis by 254% in WT mice compared to mice treated with vehicle (FIG. 1G).

### Example 2

### Endothelial-Derived EETs Stimulate Primary Tumor Growth Via Enhanced Angiogenesis

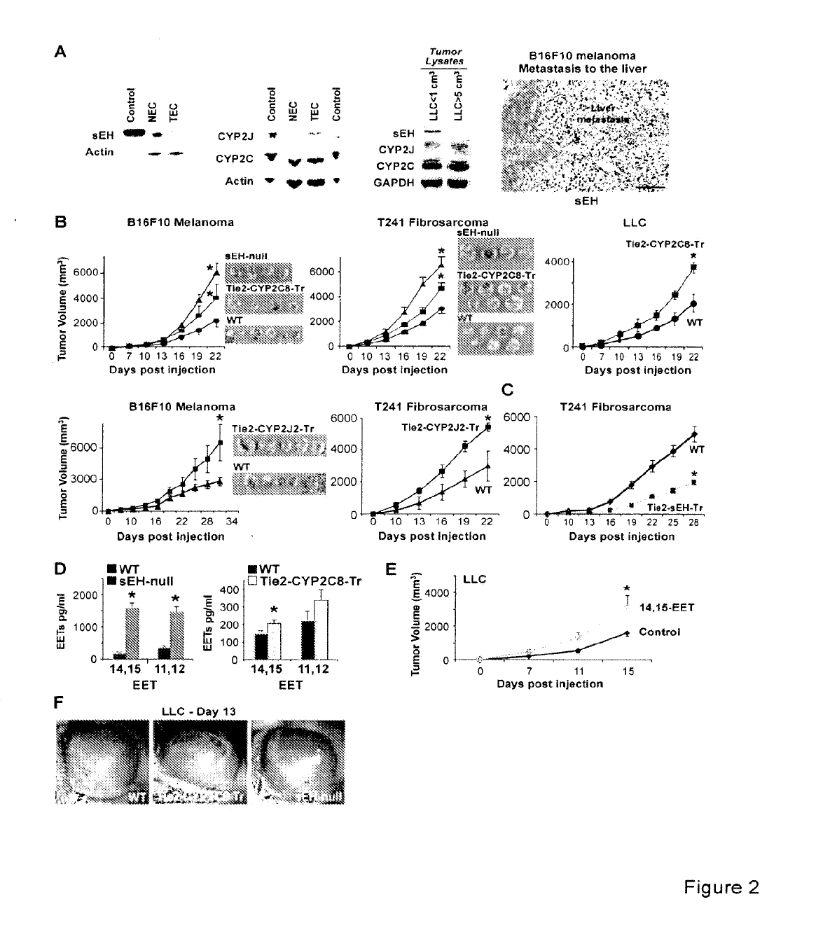

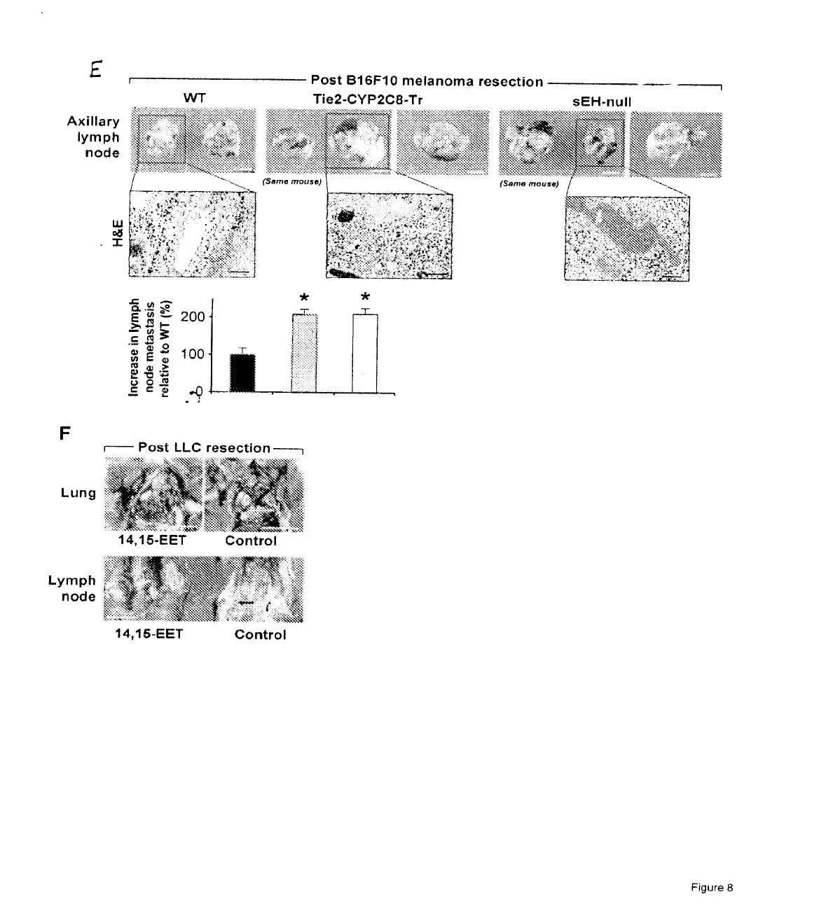

Isolated tumor endothelial cells and normal “quiescent” endothelial cells (A. C. Dudley et al., Cancer Cell 14, 201 2008) were analyzed for expression of sEH, CYP2C, and CYP2J proteins. While CYP2C and CYP2J levels were similar in the two populations of endothelial cells, a significant decrease in sEH was observed in tumor endothelial cells in comparison to normal endothelial cells (FIG. 2A, FIG. 7A). In contrast, all murine tumor cell lines that examined expressed sEH, CYP2C, and CYP2J in vitro, except for Lewis lung carcinoma (LLC) which appeared to be CYP2J-negative (FIG. 7A). Examination of LLC tumor lysates revealed that the expression of sEH, but not CYPs, decreased with tumor progression (FIG. 2A). Similarly, sEH expression was suppressed in liver metastasis of B16F10 melanoma tumors compared to adjacent normal liver tissue (FIG. 2A).

To examine the expression patterns of sEH, CYP2J and CYP2C in the tumor stroma, subcutaneous LLC and B16F10 melanoma tumors were implanted into mice. Immunohistochemistry showed that both tumor endothelial cells and pericytes expressed sEH (FIG. 7A). Immunoblotting revealed that CYP2C was also expressed in tumor endothelial cells isolated by flow cytometry (FIG. 7A). CYP2J was expressed in both tumor endothelial cells and in infiltrating inflammatory cells (FIG. 7A). Furthermore, CYP2J was localized to the endothelium in human hepatocellular carcinoma and human neuroblastoma sections (FIG. 7B).

Whether EETs could stimulate primary tumor growth was established using the genetically altered mice. A dramatic increase in the growth of B16F10 melanoma, T241 fibrosarcoma, and LLC in Tie2-CYP2C8-Tr, Tie2-CYP2J2-Tr and sEH-null mice compared to WT mice was observed, suggesting that endothelial EETs promote primary tumor growth (FIG. 2B). Conversely, Tie2-sEH-Tr mice exhibited a 60% reduction in T241 fibrosarcoma growth compared to WT mice on day 28 post-implantation (FIG. 2C, FIG. 7C).

Plasma EETs were significantly elevated in sEH-null tumor bearing mice on day 22 post-injection of T241 fibrosarcoma (FIG. 2D). 14,15-EET was also significantly increased in plasma of Tie2-CYP2C8-Tr on day 16 post-injection of LLC (FIG. 2D). The changes in eicosanoid levels were selective for epoxyeicosanoids in that PGE₂, 6-keto PGF_{2α} (stable PGI₂ metabolite), PGD₂ and several hydroxyeicosatetraenoic acid (HETE) regioisomers were not significantly altered in these models (data not shown). Since plasma EET levels and primary tumor growth were increased in genetically altered mice and exogenous EET was sufficient to promote tissue growth, we reasoned that exogenously administrated EETs might also promote primary tumor growth in WT mice. Indeed, systemic administration of 14,15-EET by osmotic mini-pump significantly accelerated primary LLC tumor growth (FIG. 2E). Collectively, these data demonstrate the necessary and sufficient role of endothelial EETs in primary tumor growth.

To determine whether tumor angiogenesis contributed to the increase in primary tumor growth in the genetically altered mice, the number of endothelial cells in tumors was analyzed by flow cytometry and immunohistochemical detection of CD31. Immunohistochemical studies showed an increase in CD31-positive cells in B16F10 tumors on day 22 post-injection in Tie2-CYP2C8-Tr, Tie2-CYP2J2-Tr and sEH-null mice relative to WT mice (FIG. 7D). Likewise, flow cytometry revealed a significant increase in CD31-positive endothelial cells in LLC tumors from the Tie2-CYP2J2-Tr mice compared to WT mice on day 22 post-injection (FIG. 7E). Accordingly, a corneal tumor angiogenesis assay revealed a dramatic stimulation of tumor-dependent angiogenesis in Tie2-CYP2C8-Tr and sEH-null mice on day 13 post-LLC injection (FIG. 2F).

### Example 3

### Endothelial-Derived EETs Trigger Massive Metastasis of Primary Tumors

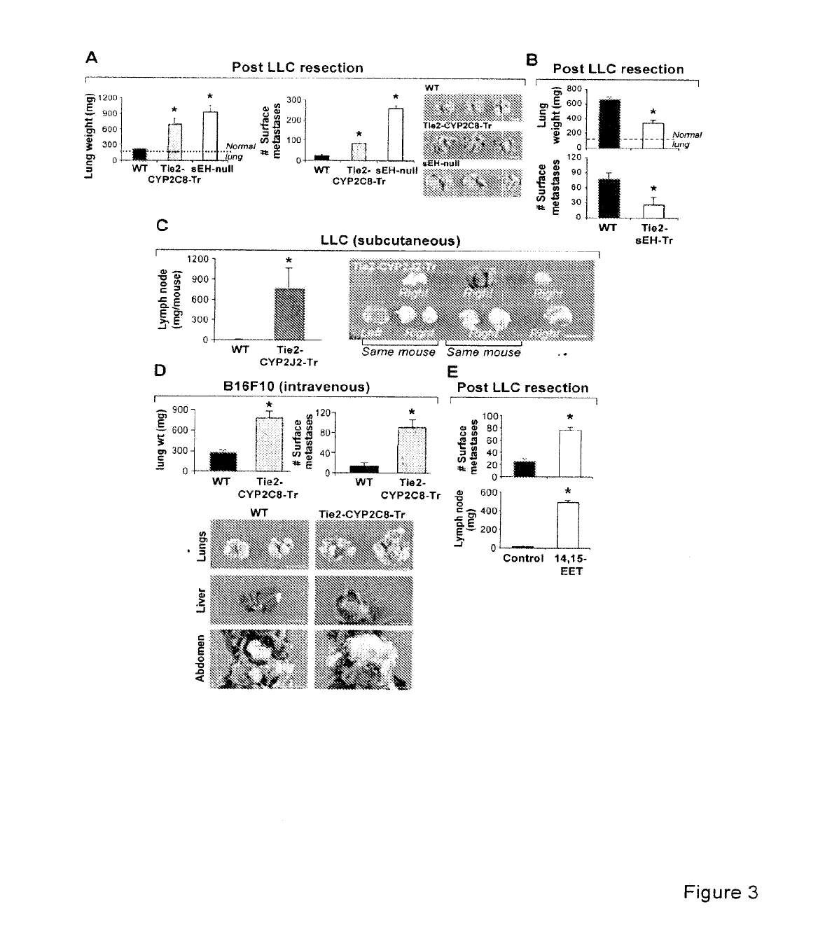

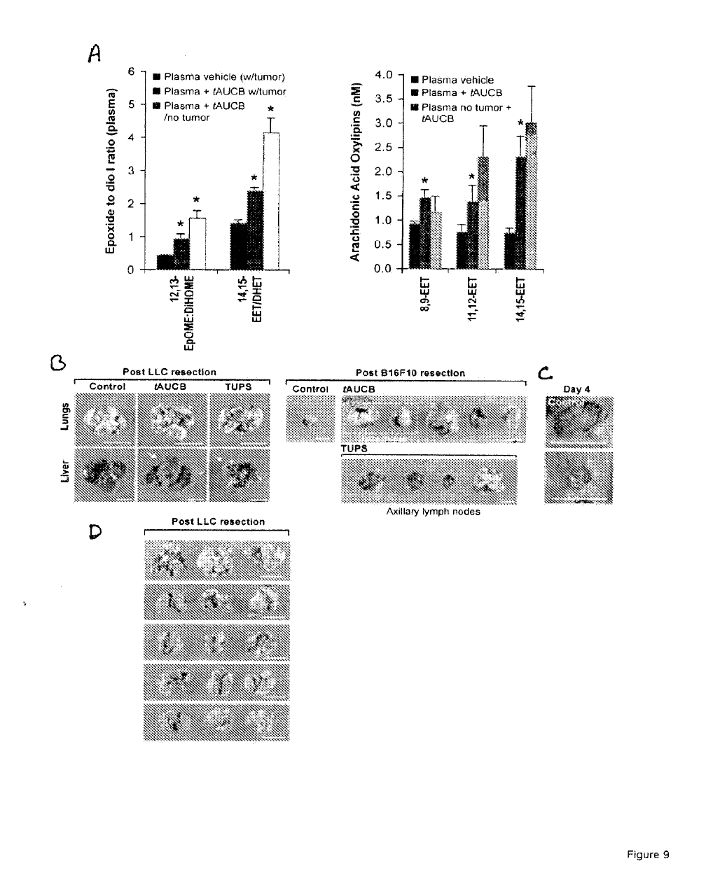

While overexpression of CYP2J2 in tumor cells has been reported to promote metastasis (J. G. Jiang et al., Cancer Res 67, 6665 2007), this effect could be due to its direct pleiotropic effects on tumor cells, including stimulation of cell growth and migration. The role of non-cell autonomous effects of endothelial-derived EETs in tumor metastasis was unknown. Whether EETs promote spontaneous metastatic growth (as opposed to induction of metastatic tumors by intravenous injection of tumor cells) was investigated using a well-established model in which resection of a primary tumor reproducibly stimulates growth of distant metastasis 14-17 days post-resection (D. Panigrahy et al., J. Clin. Invest. 110, 923 2002, M. S. O'Reilly et al., Cell 79, 315 1994). In this model, removal of the primary tumor is thought to reduce circulating, tumor-derived angiogenesis inhibitors which in turn may activate previously dormant metastases (D. Panigrahy et al., J. Clin. Invest. 110, 923 2002, M. S. O'Reilly et al., Cell 79, 315 1994). Autopsies of moribund mice 10 days after resection of primary LLC revealed a dramatic increase in lung weight and in surface lung metastases in both Tie2-CYP2C8-Tr and sEH-null mice compared with WT mice (FIG. 3A). In sEH-null mice, the normal lung tissue was completely replaced by invasive metastatic lesions. Tie2-CYP2C8-Tr and sEH-null mice also exhibited spontaneous liver and kidney metastasis (FIG. 8A). In contrast, there was a 50% decrease in the number of lung metastases in Tie2-sEH-Tr mice when compared to WT mice (FIG. 3B, 8B). These observations indicate that EETs are sufficient to stimulate spontaneous metastasis following resection of a primary tumor in genetically altered mice and are essential for the normally observed metastatic rate in WT mice.

Even without resection of the primary tumor, LLC metastases were observed in axillary lymph nodes and lungs of 100% of the Tie2-CYP2J2-Tr mice by day 22 post-injection (FIG. 3C, 8C). Hematoxylin and eosin stained sections of the axillary lymph nodes showed the presence of invading LLC tumor cells (FIG. 3D). To determine if the pro-metastatic effect of endothelial-derived EETs was limited to the LLC model, B16F10 melanoma cells were injected via the tail vein into Tie2-CYP2C8-Tr mice. In this common (non-spontaneous) hematogenic metastasis model, B16F10 cells exclusively colonize the lung and produce pulmonary metastases (R. S. Parhar, P. K. Lala, J Exp Med 165, 14 1987). However, in the Tie2-CYP2C8-Tr mice B16F10 melanoma cells produced macroscopic metastasis not only in lung but also in liver and abdomen (FIG. 3D). This is of interest because liver metastasis is commonly only achieved by directly injecting B16F10 melanoma cells into the portal or splenic vein. In parallel experiments, when primary subcutaneous B16F10 tumors were resected to trigger distant metastasis (as in the LLC model), there was a large (3-fold) increase in axillary lymph node metastasis in both Tie2-CYP2C8-Tr and sEH-null mice 17 days post B16F10 melanoma resection (FIG. 8E). This suggests that macro-metastatic tumor growth stimulated by EETs is independent of tumor type. Systemic administration of 14,15-EET via osmotic mini-pumps in WT mice at the time of LLC tumor resection stimulated a 3-fold increase in the number of surface lung metastases compared to vehicle-treated controls and led to an induction of liver, kidney and distant lymph node metastasis 12 days after resection of the primary LLC tumor (FIG. 3E, 8F).

### Example 4

### Pharmacological Manipulation of EET Levels

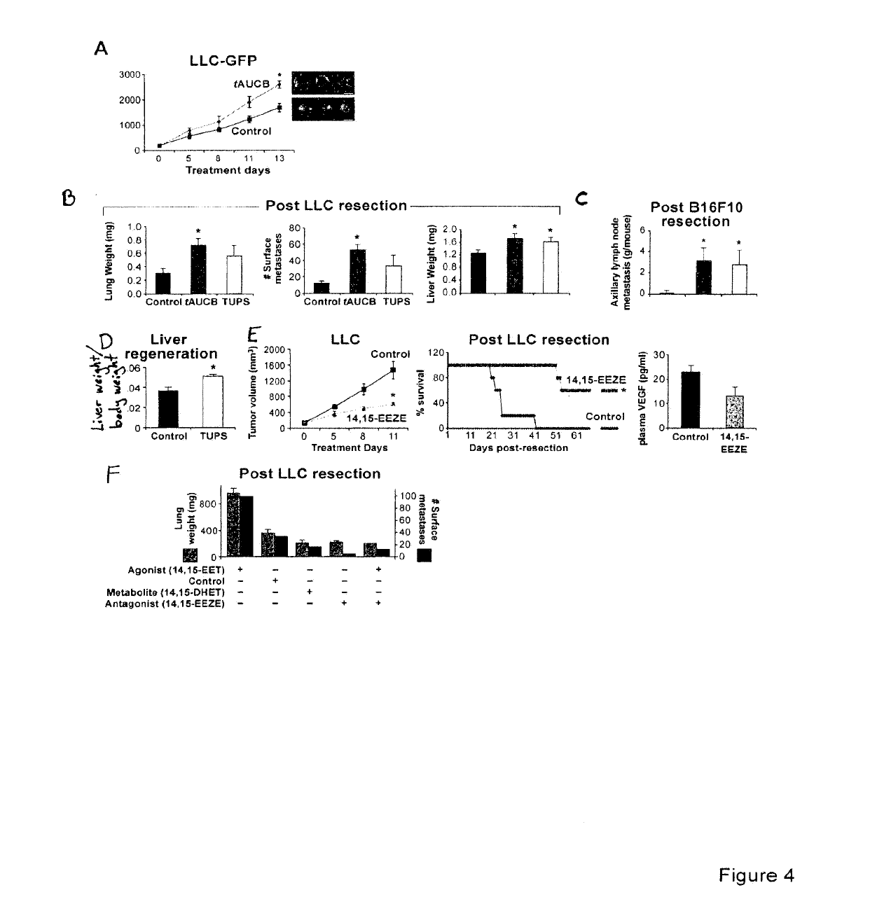

To confirm the observed effects of exogenous 14,15-EET, and to establish the clinical relevance of pharmacological EET modulation, the effect of EET modifying drugs was characterized in non-neoplastic tissue growth, primary tumor growth and metastasis models. sEH inhibitors, which increase EETs by inhibiting sEH are currently being tested in clinical trials for treatment of hypertension (J. D. Imig, B. D. Hammock, Nat Rev Drug Discov 8, 794 2009, S. H. Hwang, H. J. Tsai, J. Y. Liu, C. Morisseau, B. D. Hammock, J Med Chem 50, 3825 2007). The effect of pharmacological inhibition of sEH was investigated using t-AUCB and the structurally dissimilar TUPS. Treatment of mice with t-AUCB accelerated primary LLC-GFP tumor growth (FIG. 4A), and analysis of the plasma from tumor-bearing t-AUCB-treated mice confirmed the presence of an increase in EETs compared to vehicle (FIG. 9A). Immunohistochemical analysis of VEGF and GFP (which mark tumor cells) revealed an increase in the number of tumor cells expressing VEGF and a marked increase in microvessel density in t-AUCB-treated LLC-GFP tumors (data not shown). Systemic administration of either t-AUCB or TUPS dramatically stimulated spontaneous lung, liver and axillary lymph node metastasis in two different murine tumor models (LLC and B16F10 primary tumor resection) in WT mice (FIG. 4C, 9B). Similarly, administration of TUPS increased liver regeneration by 38% and significantly accelerated wound healing when compared to vehicle-treated mice (FIG. 4D, 9C).

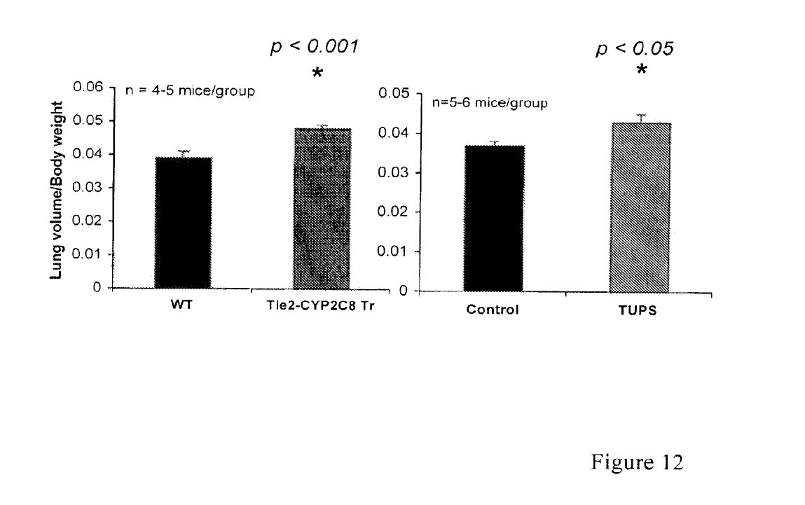

As shown herein, epoxyeicosatrienoic acids (EETs) stimulate angiogenesis, in part via the VEGF signaling network. Since compensated lung growth is dependent on VEGF-induced angiogenesis, it was hypothesized that endothelial cells (ECs) stimulate lung growth via production of EETs. To confirm the effects of CYP2C8 overexpression, EET levels were increased by inhibition of soluble epoxide hydrolase (sEH) following left penumonectomy. The sEH inhibitor TUPS stimulated lung growth/body weight (p<0.05) on day 4 post-pneumonectomy compared to vehicle-treated mice (p<0.05) (FIG. 12). In contrast, there was no significant change in baseline lung volume/body weight ratio after sham operation.

Conversely, mice with established LLC tumors treated with the putative EET receptor antagonist 14,15-EEZE demonstrated reduced primary LLC growth, prolonged survival in the LLC resection metastasis model and reduced plasma VEGF levels (FIG. 4E). To determine if an EET-antagonist could prevent EET-induced metastasis, the EET antagonist 14,15-EEZE was co-administered with 14,15-EET following LLC primary tumor resection. The EET antagonist reduced lung metastasis and prevented macroscopic liver and lymph node metastasis typically induced by 14,15-EET (FIG. 4F, 9D).

### Example 4

### Mode of Action

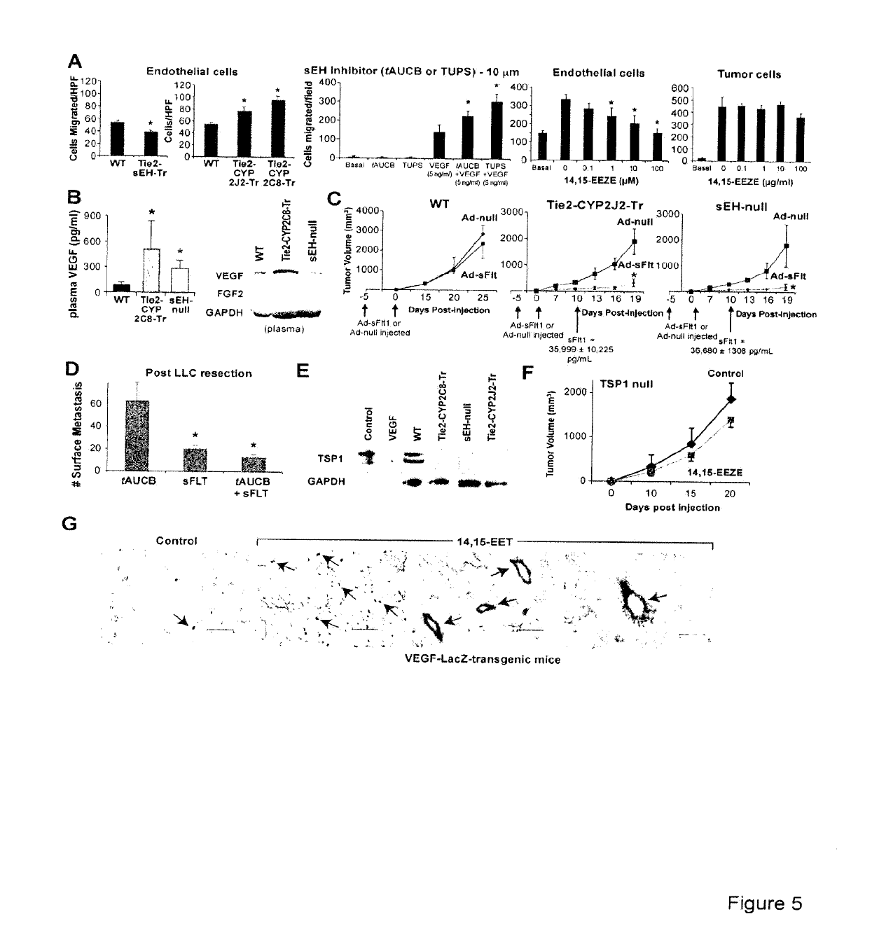

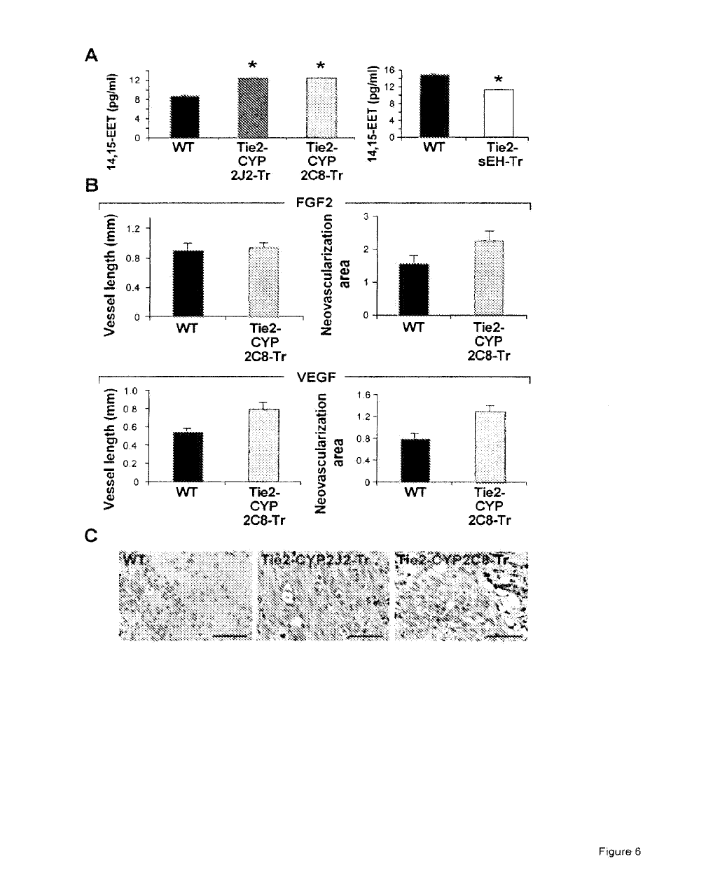

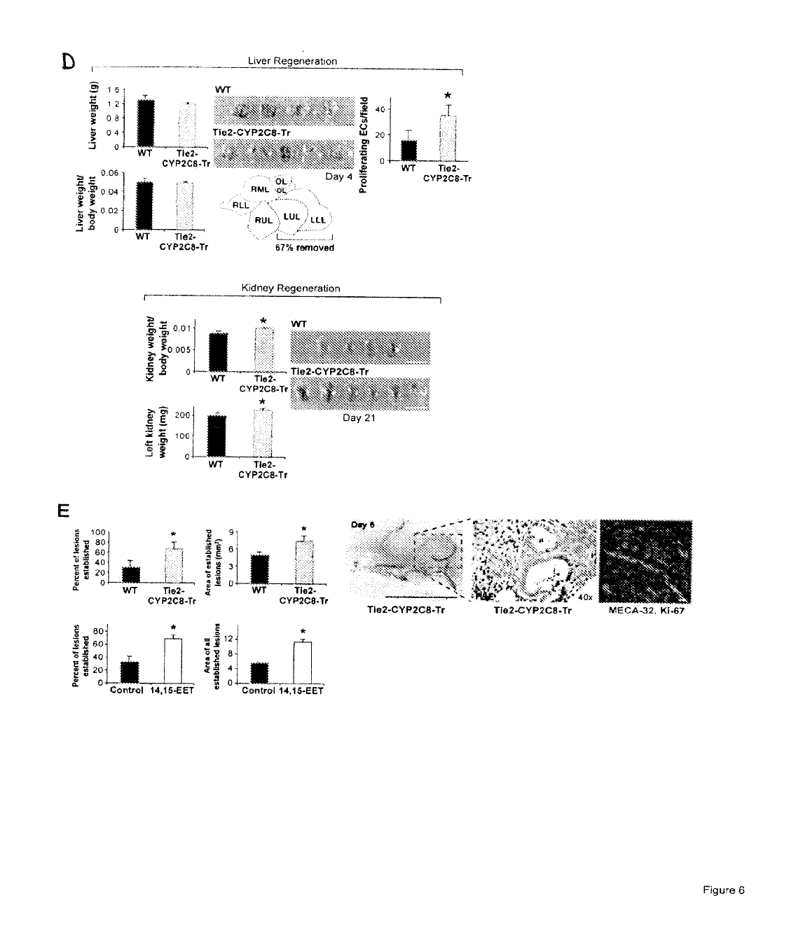

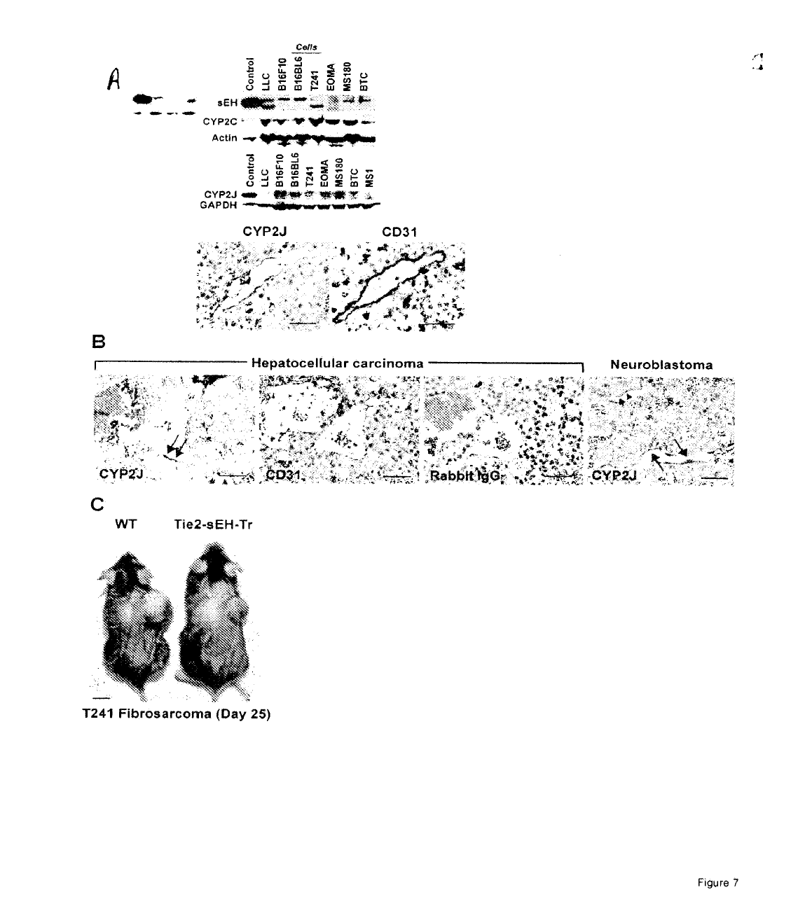

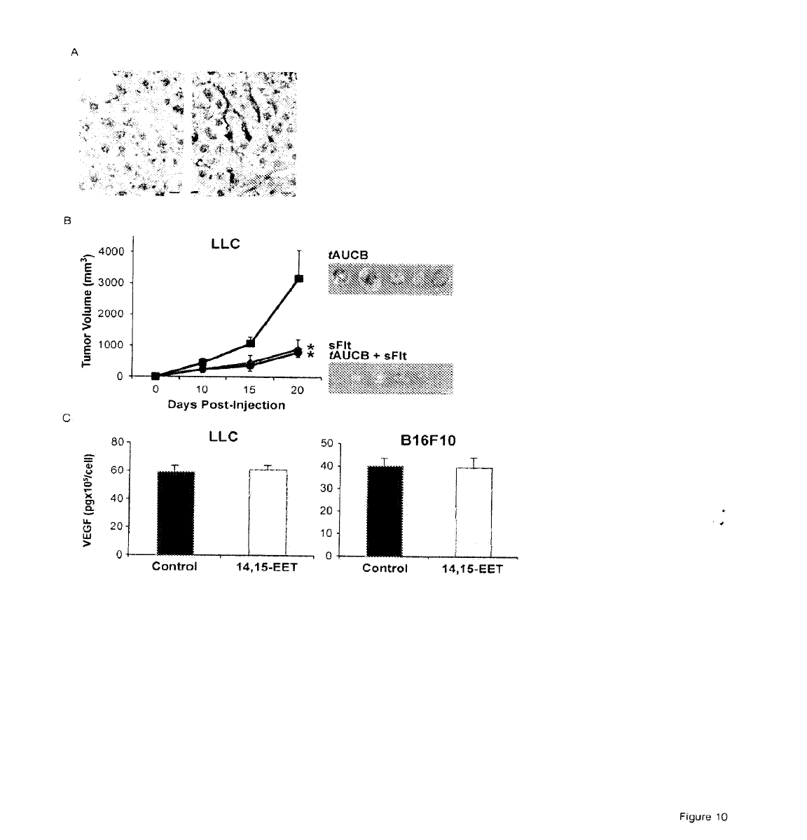

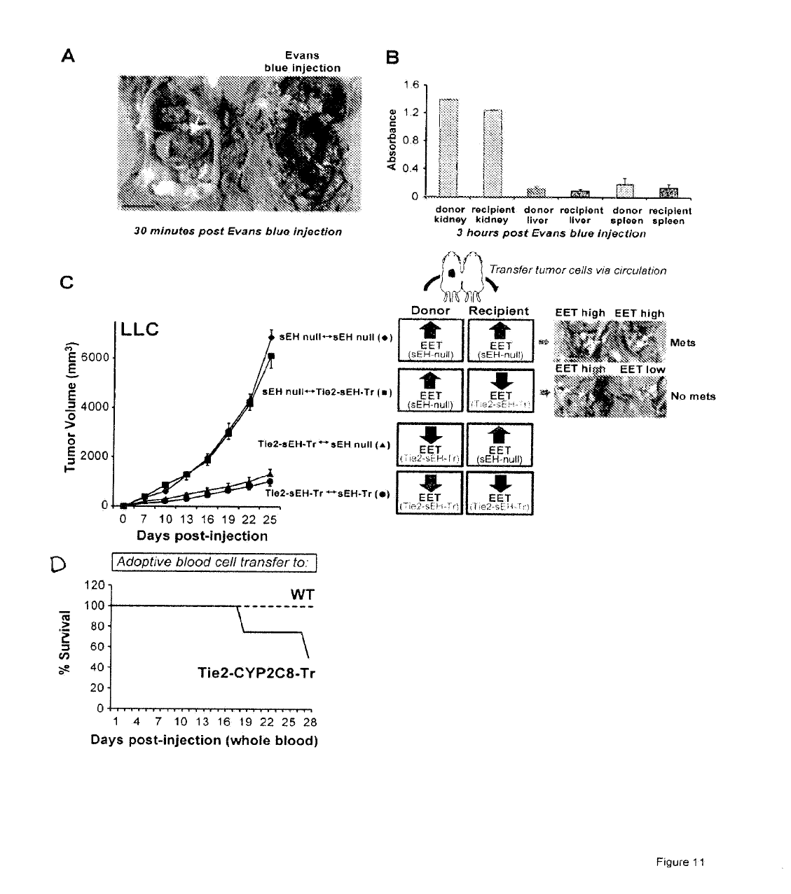

Endothelial cells isolated from the aortas of Tie2-sEH-Tr mice, which have endothelial-specific staining of sEH (FIG. 10A), exhibited decreased migration on collagen substrates when compared to aortic endothelial cells from WT mice (FIG. 5A). In contrast, endothelial cells from Tie2-CYP2J2-Tr and Tie2-CYP2C8-Tr mice exhibited increased migration relative to WT endothelial cells (FIG. 5A). t-AUCB and TUPS had no significant effects on basal endothelial migration, but stimulated VEGF-mediated endothelial migration 2- to 3-fold (FIG. 5A). In contrast, the putative EET-receptor antagonist 14,15-EEZE inhibited endothelial migration in a dose-dependent fashion but had no significant effect on migration of LLC tumor cells (FIG. 5A).

Both Tie2-CYP2C8-Tr and sEH-null mice exhibited a significant increase in plasma levels of VEGF but not FGF2, when compared to WT mice (FIG. 5B). This is consistent with results from the corneal assays showing increased VEGF- but not FGF2-stimulated angiogenesis in these mice (FIG. 6B). To determine whether VEGF-stimulated angiogenesis plays a functional role in EET-mediated tumor growth, VEGF was depleted by expression of a soluble form of VEGF receptor 1 (sFlt) using an adenoviral delivery system (C. J. Kuo et al., Proc Natl Acad Sci USA 98, 4605 2001). In WT mice, which have low systemic VEGF levels, VEGF depletion had no significant effect on primary B16F10 melanoma growth (FIG. 5C). In contrast, in Tie2-CYP2J2-Tr and sEH-null mice, where EET and VEGF levels were high, VEGF depletion suppressed B16F10 melanoma tumor growth by up to 80% at day 19 post-tumor injection (FIG. 5C). Tumor tissues contained high levels of VEGF; however, when VEGF was depleted by sFlt, the tumor microvasculature, visualized by MECA32, was discontinuous, reflecting the immature vessel phenotype observed in the absence of VEGF (R. T. Tong et al., Cancer Res 64, 3731 2004) (data not shown). In fact, t-AUCB was unable to promote tumor growth and metastasis in mice depleted of VEGF by sFlt (FIGS. 5D and 10B).

B16F10 tumors in mice expressing sFlt eventually ‘escaped’ VEGF depletion, suggesting that other regulators of tumor angiogenesis, such as thrombospondin-1 (TSP1), which is an angiogenesis inhibitor (M. Streit et al., Am J Pathol 155, 4411999), may also play an important role. Indeed, plasma from tumor-bearing Tie2-CYP2C8-Tr, Tie2-CYP2J2-Tr, and sEH-null mice exhibited a pronounced reduction in the levels of TSP1 at day 13 post-injection when compared to WT mice (FIG. 5E), suggesting that this effect may contribute to the tumor promoting activity of EETs. To evaluate the potential role of TSP1 as a mediator of EET-induced tumorigenesis, TSP1-deficient (TSP1 null) mice were treated with the EET antagonist 14,15-EEZE whereupon tumor suppression by the EET antagonist was significantly diminished, by 62% in WT mice (FIG. 4E) to 24% in TSP1 null mice (FIG. 5F).

14,15-EET had no significant effect on VEGF production by LLC or B16F10 tumor cells in vitro (FIG. 10C). However, analysis of LLC tumors in VEGF-LacZ-Tr mice in which the LacZ reporter was introduced into the VEGF locus (A. S. Maharaj, M. Saint-Geniez, A. E. Maldonado, P. A. D'Amore, Am J Pathol 168, 639 2006). Revealed that the endothelium and stromal fibroblasts of LLC tumors grown in VEGF-LacZ-Tr mice systemically treated with 14,15-EET stained positively for the LacZ product, β-galactosidase whereas this marker for VEGF production was absent in the stroma of size-matched control tumors of non-treated mice (FIG. 5G).

As shown herein, epoxyeicosatrienoic acids (EETs) stimulate angiogenesis, in part via the VEGF signaling network. Since compensated lung growth is dependent on VEGF-induced angiogenesis, it was hypothesized that endothelial cells (ECs) stimulate lung growth via production of EETs. Transgenic (Tg) mice with EC-specific overexpression of CYP2C8 (Tie2 promoter-driven) were used to study the consequence of increased EETs on lung growth following left pneumonectomy. To confirm the effects of CYP2C8 overexpression, EET levels were increased by inhibition of soluble epoxide hydrolase (sEH). EC-specific overexpression of CYP2C8 promoted contralateral lung growth following unilateral pneumectomy. Lung volume/body weight on day 4 post-pneumonectomy were increased by 23% (p<0.001) in Tg mice compared to WT mice (FIG. 12). In contrast, there was no significant change in baseline lung volume/body weight ratio after sham operation.

### Example 5

### Parabiosis

To determine whether endothelial-derived EETs facilitate cancer cell dissemination at the primary tumor site (by promoting extravasion and migration) or at the metastatic site (by promoting homing, colonization, dormancy escape, survival, etc), a parabiosis model with a shared circulatory system (A. D. Soutter, J. Ellenbogen, J. Folkman, J Pediatr Surg 29, 1076, 1994, I. M. Conboy et al., Nature 433, 760 2005, T. Nakamura et al., Neoplasia 9, 979 2007, G. Pietramaggiori et al., J Invest Dermatol 2009). between tumor-bearing “donor” mice with high EET levels (Tie2-CYP2C8-Tr or sEH-null) conjoined to non-tumor bearing “recipient” mice with normal or low endothelial EET levels (WT or Tie2-sEH-Tr, respectively) was utilized. Other configurations served as controls. The sharing of humoral factors in the parabiotic circulation was confirmed using Evans blue dye as a tracer (FIG. 11A, 11B). Results of the experiments are summarized in Table 1 and FIG. 11C. In the control configuration in which two “high EET” mice were parabiosed but only one carried the primary tumor, liver, lung and lymph node metastases occurred in both parabionts, demonstrating that parabiosis itself did not interfere with EET-promoted metastasis (Table 1, cases b, c and i). Moreover, the genotype of the tumor-bearing donor mouse determined the growth of the primary tumor, regardless of the genotype of the recipient mouse. Specifically, sEH-null (high EET) recipients could not “rescue” the tumor phenotype of Tie2-sEH-Tr (low EET) donors (Table 1, case f). It is possible that the high EETs in the plasma of sEH null mice, which is shared by both partners, was not sufficient to rescue tumor growth in the Tie2-sEH-Tr-mice where the sEH enzyme keeps EETs in the endothelium low. In the key configuration, a low EET recipient attached to a high EET donor failed to produce metastasis (Table 1, cases j and k), demonstrating unequivocally that a high EET-producing endothelium is critical at the metastatic site for lung, liver and lymph node metastases to occur. Importantly, adoptive transfer of whole blood from low EET recipient parabionts (Tie2-sEH-Tr), which exhibited no metastasis, caused metastatic disease when inoculated into non-parabiotic high EET (Tie2-CYP2C8-Tr) mice, confirming that the low EET parabiont had circulating tumor cells. Indeed, 28 days post-injection, 100% of the WT recipients of blood transfer survived while 50% of the Tie2-CYP2C8-Tr blood transfer recipients died of metastasis (FIG. 11D). Immunofluorescent staining of histological sections of. tumors removed from the high EET parabionts showed increased tumor EC proliferation and increased VEGF production (data not shown).

All patents and other publications identified are expressly incorporated herein by reference for the purpose of describing and disclosing, for example, the methodologies described in such publications that might be used in connection with the methods described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 1
Parabiosis shows that EET-stimulated tumor growth depends on genotype of
tumor-bearing parabiont (donor) (upper panel) and that metastasis requires
EET-producing endothelium at the metastatic site in the recipient parabiont
(lower panel). Parabiosis was performed for 5 pairs per configuration and
average tumor volume ± sem determined. *p < 0.05 vs cases a, f, and g
(upper panel); *p < 0.05 vs cases h, j, and k (lower panel).

| | |
|---|---|
| Parabiotic “Donor” | “Recipient” |
| (=site of primary tumor) | (site) |

Parabiosis “Donor”/“Recipient” constellations on primary tumor growth (LLC)

| | | | Primary tumor volume | |
|---|---|---|---|---|
| a | WT | WT | 1565 ± 170 | mm³ |
| b | sEH-null → EET high | sEH-null → EET high | 6869 ± 311 | mm³* |
| c | Tie2-CYP2C8-Tr | Tie2-CYP2C8-Tr | 5632 ± 1004 | mm³* |
| | → EET high | → EET high | | |
| d | sEH-null → EET high | Tie2-sEH-Tr → EET low | 6123 ± 505 | mm³* |
| e | Tie2-CYP2C8-Tr | WT → EET low | 5046 ± 526 | mm³* |
| | → EET high | | | |
| f | Tie2-sEH-Tr → EET low | sEH-null → EET high | 1333 ± 183 | mm³ |
| g | Tie2-sEH-Tr | Tie2-sEH-Tr | 1031 ± 186 | mm³ |
| | →EET very low | →EET very low | | |

Parabiosis “Donor”/“Recipient” constellations on spontaneous LLC metastasis

| | | | |
|---|---|---|---|
| | | | # surface |
| | | | lung metastasis in recipient |
| h | WT | WT | No metastasis |
| i | Tie2-CYP2C8-Tr | Tie2-CYP2C8-Tr | 27 ± 4; 3/4 liver mets; |
| | → EET high | → EET high | 3/4 lymph node mets* |
| j | Tie2-CYP2C8-Tr | WT → EET low | No metastasis |
| | → EET high | | |
| k | Tie2-CYP2C8-Tr | Tie2-sEH→ EET very low | No metastasis |
| | → EET high | | |

What is claimed:

1. A method of promoting cell proliferation in a tissue in need thereof, the method comprising contacting said tissue with a therapeutically effective amount of a soluble epoxide hydrolase inhibitor (sEHi).

2. The method of claim 1, wherein angiogenesis is enhanced by the contacting.

3. The method of claim 1, wherein endothelial cell migration is enhanced by the contacting.

4. The method of claim 2, wherein the sEHi inhibits the activity of a soluble epoxide hydrolase (sEH) or inhibits the expression of a sEH gene in the tissue.

5. The method of claim 1, wherein the sEHi is selected from a group consisting of a small molecule, nucleic acid, nucleic acid analogue, protein, antibody, peptide, aptamer and variants or fragments thereof.

6. The method of claim 1, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea (TUPS).

7. The method of claim 1, wherein the sEHi is an antibody which can specifically bind to and inhibit sEH activity.

8. The method of claim 1, wherein the sEHi is an anti-sEH oligonucleotide, an antisense oligonucleotide to the sEH gene, an siRNA to sEH gene, or a locked nucleic acid that anneals to the sEH gene, wherein the expression of the sEH gene is inhibited.

9. The method of claim 1, wherein the method is applied in the context of neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.

10. A method of promoting angiogenesis in a tissue in need thereof, the method comprising contacting said tissue with a therapeutically effective amount of a soluble epoxide hydrolase inhibitor (sEHi).

11-12. (canceled)

13. The method of claim 10, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea (TUPS).

14-37. (canceled)

38. A method of promoting tissue growth or regeneration, the method comprising contacting said tissue with a therapeutically effective amount of a soluble epoxide hydrolase inhibitor (sEHi), whereby tissue growth or regeneration is enhanced relative to tissue growth or regeneration in the absence of said sEHi.

39. A method of promoting tissue growth or regeneration in a subject, the method comprising administrating a therapeutically effective amount of a soluble epoxide hydrolase inhibitor (sEHi), whereby tissue growth or regeneration is enhanced relative to tissue growth or regeneration in the absence of administrating said sEHi.

40. The method of claim 38, wherein angiogenesis is enhanced by the contacting.

41-42. (canceled)

43. The method of claim 38, wherein the sEHi is trans-4-[4-(3-adamantan-1-yl-ureido)-cyclohexyloxy]-benzonic acid (tACUP) or 1-(1-methanesulfonyl-piperidin-4-yl)-3-(4-trifluoromethoxy-phenyl)-urea (TUPS).

44-45. (canceled)

46. The method of claim 38, wherein the method is applied in the context of promoting wound healing, neuronal growth, protection or repair, tissue repair, tissue regeneration, fertility promotion, cardiac hypertrophy, treatment of erectile dysfunction, modulation of blood pressure, revascularization after disease or trauma, tissue grafts, or tissue engineered constructs.
